(19) United States
(12) Patent Application Publication
(10) Pub. No.: US 2010/0080757 A1
(43) Pub. Date: Apr. 1, 2010

(54) METHOD OF DETECTING ABNORMAL TISSUE

(76) Inventors: John R. Haaga, Chagrin Falls, OH (US); Nicholas Stowe, Bay Village, OH (US); Agata A. Exner, Westlake, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 11/815,172

(22) PCT Filed: Feb. 8, 2006
(86) PCT No.: PCT/US06/04378

§ 371 Date: May 8, 2009

Related U.S. Application Data
(60) Provisional application No. 60/650,779, filed on Feb. 8, 2005.

(51) Int. Cl. A61K 49/06; A61K 49/00; A61K 49/04; A61K 49/22

(52) U.S. Cl. 424/9.3; 424/9.1; 424/9.4; 424/9.5

(57) ABSTRACT

A method for detecting abnormal biological tissue includes administering a vasoactive agent and determining the rate of blood flow in a tissue of interest.

### RELATED APPLICATION

The present application claims priority from U.S. Provisional Application Ser. No. 60/650,779 filed Feb. 8, 2005, which is herein incorporated by reference in its entirety.

### TECHNICAL FIELD

The present invention relates to diagnostic imaging, and more particularly to the use of diagnostic imaging in visualizing tissue abnormalities.

### BACKGROUND OF THE INVENTION

Angiogenesis occurs in the healthy body for healing wounds and for restoring blood flow to tissues after injury or insult. Typically, normal vascularity develops with the intent of providing nutrients to body tissues, and normal vessels evolve so that equilibrium is established between vessel growth and cellular demands. When angiogenic growth factors are produced in excess of angiogenesis inhibitors, for example, the balance is tipped in favor of blood vessel growth. When inhibitors are present in excess of stimulators, angiogenesis is stopped. Moreover, the normal, healthy body maintains a perfect balance of angiogenesis modulators.

In many disease states, the body loses control over angiogenesis. In the case of cancer, excessive angiogenesis occurs when diseased cells produce abnormal amounts of angiogenic growth factors, which can overwhelm the effects of natural angiogenesis inhibitors. This uncontrolled process can lead to hyper-proliferation of the cells surrounding the newly fowled vessels, and ultimately can lead to tumor formation. Early detection of uncontrolled angiogenesis is critical to reducing morbidity and mortality rates associated with various cancers. Some of the most commonly used detection modalities are based on nuclear, ultrasound, and X-ray imaging technologies.

Despite advancements in clinical imaging technology, clear and unfailing methods of identifying malignant tissue from benign and normal tissue remain elusive. Currently accepted protocols range from manually enhancing regions on computed tomography (CT) and MRI scans, to injection of radiotracers for nuclear imaging. All of these methods still require a follow-up biopsy to identify the pathology of the tissue in question. In a related problem, follow-up imaging of procedures, such as tumor radiofrequency ablation also fail to demonstrate procedure success, and remains dependent on tissue biopsy should uncertainty arise.

### SUMMARY OF THE INVENTION

The present invention relates to a method of detecting abnormal biological tissue. The abnormal biological tissue can include a lesion, such as neoplastic tissue. Additionally, the abnormal biological tissue can include tissue that is inflamed and/or scarred as a result of post-clinical treatment.

In the method, an amount of a vasoactive agent effective to modify the blood flow rate in a tissue of interest is administered to a subject. Following administration of the vasoactive agent, the blood flow rate in the tissue is determined as being increased or decreased in comparison to the blood flow rate in normal tissue.

In an aspect of the invention, the vasoactive agent includes a vasodilatory agent and/or a vasoconstrictive agent. Examples of vasoactive agents include carbonic anhydrase inhibitors, caffeine citrate, organic nitrates, glyceryl trinitrate, pentaerythritol tetranitrate, hydralazine, sildenafil citrate, minoxidil, diazoxide, sodium nitroprusside, isosorbide dinitrate, isosorbide mononitrate, cilostazol, papaverine, dipyridamole, oxyfedrine hydrochloride (HCl), diltiazem HCl, tolazoline HCl, hexobendine, bamethan sulfate, sulfonamide derivatives, phenylephrine HCl, pitressin, pseudoephedrine, angiotensin, vasopressin, levonordefrin, epinephrine, naphazoline nitrate, tetrahydrozoline HCl, oxymetazoline HCl, tramazoline HCl, lypressin, and combinations thereof.

In another aspect of the invention, the blood flow rate in the tissue of interest is determined by generating at least one first image of the tissue of interest prior to administration of the vasoactive agent, generating at least one second image of the tissue of interest after administration of the vasoactive agent, and comparing first images and the second images. The first image and the second image can be compared to identify any local variations in the change in signal intensity resulting from vascular volume changes induced by the vasoactive agent. The first images and the second images can be generated by at least one of computed tomography (CT), magnetic resonance (MR), ultrasound, and X-ray.

Another aspect of the invention relates to a method of detecting abnormal biological tissue in a patient. In the method, the blood flow rate in a tissue of interest is determined prior to administration of a vasoactive agent. An amount of the vasoactive agent effective to modify the blood flow rate in the tissue of interest is administered. The blood flow rate in the tissue of interest is determined following administration of the vasoactive agent. The blood flow rate in the tissue of interest can be determined prior to and following administration of the vasoactive agent by imaging the tissue of interest. Images of the tissue of interest being generated by at least one of computed tomography, magnetic resonance, ultrasound, and X-ray.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a schematic flow diagram illustrating a method in accordance with an aspect of the invention;

FIG. 2 is a CT perfusion measurement in VX2 tumor (T2) and normal liver (T1) at day 28, (A) CT image, (B) Arterial perfusion (pseudo color) measurement (C) The hepaptic artery A1, portal vein (V1), and splenic blood flow inputs to the single compartment model;

FIG. 3 is a photograph illustrating the gross pathology of VX2 tumor in the liver at day 28. Tumor tissue is comprised of fibrous outer capsule and a nectrotic core;

FIG. 4 is a plot illustrating the correlation of tumor size and perfusion. Growth of VX2 tumor results in an increase in blood flow (with arterial increasing slightly slower than venous) for the first 21 days. During the last week tumor necrosis is extensive and perfusion decreases;

FIG. 5 is a chart showing the net percent change in tumor and normal liver perfusion after acetazolamide injection as measured by functional CT.

FIGS. 6 (a-c) is a photograph showing staining of liver tissue (10×). FIG. 6A is a picture showing smooth muscle cell actin staining of normal liver. FIG. 6B shows an inflammatory response in liver tissue. FIG. 6C shows hepatic tumor tissue.

FIGS. 7(a-b) are plots illustrating MRI perfusion study with the solid line showing flow in a small hepatoma and the dotted line showing flow in the normal parenchyma. FIG. 7a illustrates a baseline perfusion study showing quicker and higher enhancement of tumor vs normal liver. FIG. 7b illustrates AZ enhanced perfusion shows a slight delay in the onset of enhancement and decrease in enhancement of the tumor compared to the normal liver.

FIG. 8 is a plot illustrating FDG uptake curves in woodchuck hepatoma from two PET studies. The lower curve shows baseline FDG alone and the upper curve is derived from PET with FDG and phenylephrine. Increased flow to lesion produced increased uptake.

FIGS. 9(a-b) are baseline CT (A) and corresponding PET (B) images of lung cancer. FIGS. 9(c-d) are images illustrating Tumor after oral pseudoephedrine. The tumor has not changed size on CT but has increased PET signal. SUV increased from 4.8 to 8.8, presumably due to increased blood flow to tumor and capture of FDG.

FIG. 10 is a plot illustrating CT perfusion analysis of a biopsy proven inflammatory pseudotumor before and after acetazolamide (AZ) administration (AZ 250 mg+50 mL Optiray 320).

FIGS. 11(a-c) are MR of suspected recurrent tumor in liver without (A) and after (B) vasodilator (Diamox), and image-guided biopsy confirmation (C). In A and B, the solid line corresponds to the lesion and the “dashed” line corresponds to the normal liver.

FIG. 12 is a plot illustrating gadolinium enhancement after bolus injection of 20 cc of gadolinium veresetamide without sildenafil dosage. Intensity time curve plots intensity value against time in seconds over prostate medial and lateral lobe, normalized to adjacent internal obturator muscle, and femoral artery. In the early vascular phase the enhancement of the lobes is quite gradual and maximizes after minutes. At this time as the intensity includes both the vascular and extra-vascular space. Note the increased enhancement of the medial lobe above the lateral lobe.

FIG. 13 is a plot illustrating Gadolinium enhancement after bolus of 20 cc of gadolinium after administration of sildenafil. Versetamide 30 minutes after sildenafil. The same pulsing sequence and time intervals were used on the second exam as the one displayed in FIG. 13. The enhancement uptake in the early vascular phase is much more rapid with the vasodilator and the maximum enhancement is 73% greater in the lateral lobe than the enhanced, undilated study above. The medial lobe after silfenadil (Viagra) enhances 78% greater than the medial lobe of the enhanced undilated study. Note also there is greater vascular enhancement of the lateral lobe than the medial lobe beginning at 21 seconds after the bolus, arrow. At 72 seconds, the medial lobe enhancement increases to exceed that of the lateral lobe.

FIG. 14 is a plot illustrating gadolinium enhancement after administration of sildenafil and pseudoephedrine. Patient had oral dose of Sildenafil 25 mg, 1 hour before study and oral dose of pseudoephedrine 60 mg before injection of gadolinium contrast (20 cc of Versetamide). Early part of curve shows similar overall enhancement and separation of medial and lateral lobe enhancement as noted in FIG. 13. In the equilibration phase at 14 minutes when pseudoephedrine serum levels are increasing, there is a definite decrease in intensity due to vasoconstriction. Gastrointestinal absorption kinetics is well defined over time and is unaffected by food, drink, or any other parameters.

FIG. 15 is an MRI image of the prostate as baseline before the injection of Gadolinium. Note the almost equivalent intensity of the obturator, muscle and prostate.

FIG. 16 is an MRI image of the prostate at maximum enhancement after Gadolinium injection. No Viagra was given during this study

FIG. 17 is an MRI image of separate MRI study on different day after a dose of sildenafil 50 mg, and injection of gadolinium. There is greater enhancement of the gland than in FIG. 16.

### DETAILED DESCRIPTION

The present invention relates to a method of detecting abnormal biological tissue and more particularly to a method of distinguishing abnormal biological tissue from normal tissue. The abnormal biological tissue can include, for example, a lesion, such as neoplastic tissue that is present in hepatic, renal, breast, pulmonary, ovarian, and prostrate tissues. The present invention may also be useful for distinguishing different tissue types resulting from post-operative treatment, for instance, tissue that is scarred and/or inflamed as a result of post-clinical treatment.

The method of the present invention is based, in part, on the inherent differences between normal and abnormal tissue vascularity. Upon administration of a vasoactive agent to a subject being treated. The generalized normal vascular system reacts to these agents while the vascular system of abnormal tissue (e.g., tumor vessels) remains unresponsive. The net effect on blood flow within abnormal tissue (e.g., tumor) vascular beds is converse to that in the normal vascular bed. A vasoconstrictor produces constriction of normal vessels increasing local vascular pressure, which then diverts blood to the abnormal tissue (e.g., tumor) vascular bed, thereby increasing flow to the abnormal tissue. A vasodilator produces general dilation of normal vessels and a “steal” effect occurs from the abnormal tissue vascular bed. The method of the present invention therefore capitalizes upon the ability of different imaging techniques to detect differential effects of vasoactive agents on normal and abnormal tissue.

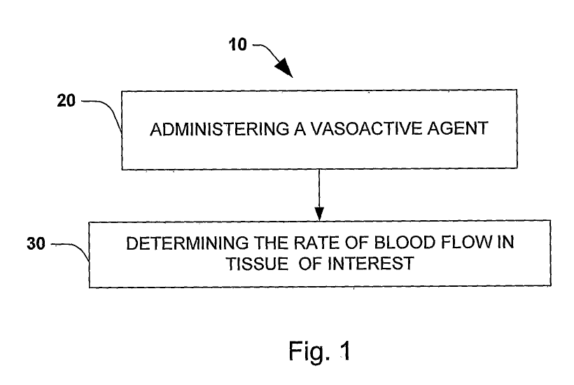

FIG. 1 is a schematic flow diagram illustrating a method 10 in accordance with the present invention. In the method 10, at 20, a vasoactive agent is administered to a patient. The vasoactive agent can include compounds that can cause constriction or dilation of blood vessels in normal tissue with an increase or decrease in blood flow to the tissue of interest. Examples of vasoactive agents include vasodilatory and vasoconstrictive agents.

Vasodilators in accordance with the present invention can include any agent that acts as a blood vessel dilator, that decreases interstitial pressure in normal tissue, and that increases vascular blood flow in normal tissue, such as by opening blood vessels by relaxing their muscular walls. One example of a vasodilator that can be use in accordance with the present invention is a carbonic anhydrase inhibitor, such as acetazolamide (AZ). Acetazolamide (AZ) is used clinically for its diuretic properties and as a vasodilatory stimulus in cerebrovascular disease (Settakis, G. et al. Eur. J. Neurol., 10(6):609-20 (2003)). Carbonic anhydrase catalyzes the conversion of carbon dioxide into carbonic acid (Taki, K. et al. Res. Commun. Mol. Pathol. Pharmacol., 103(3):240-8 (1999); Taki, K. et al. Angiology, 52(7):483-8 (2001)). The inhibition of this enzyme by carbonic anhydrase inhibitors (such as AZ) causes an increase in CO₂ (hypercapnia) and a reduction in NOX, and thus induces selective vasodilation in tissues with inherently high levels of carbonic anhydrase, including liver and kidney (Taki, K. et al. Res. Commun. Mol. Pathol. Pharmacol., 103(3):240-8 (1999); Taki, K. et al. Angiology, 52(7):483-8 (2001)). Taki et al. demonstrated that the administration of AZ produced preferential increase in blood flow to the liver (75+/−36%) and kidney (33+/−11%) without systemic effects.

Studies have also evaluated the expression of carbonic anhydrase in tumor tissue. The expression of cytosolic carbonic anhydrase in human hepatocellular carcinoma (HCC) has been studied. Histological samples from 60 cases of HCC and 10 cases of cholangiocarcinoma were compared to ten normal liver samples. The results showed that carbonic anhyrase mRNA expression was reduced in tumor tissue as compared to normal tissue. It was stated that the reduction of the three carbonic anhydrase isozymes (CAI, CAII, and CAIII) might promote tumor cell motility, growth and metastases. Although these measurements were attributed to hepatocytes, the wide spread presence and contractile nature of sinusoidal pericytes has been well documented. It is thus plausible that these pericytes also lack the normal carbonic anhydrase inhibitor. Sinusoidal pericytes, which are widespread in liver parenchyma, have significant contractile properties. The reduction of carbonic anhydrase in tumors should result in a large discrepancy between normal and tumor tissue perfusion that can then be translated into signal intensity changes on image studies.

Another example of a vasodilator that can be used in accordance with the present invention is caffeine. Caffeine, a central nervous system stimulant, is sparingly soluble in water, but in the presence of citric acid forms caffeine citrate salt in solution, or caffeine citrate (available as CAFCIT®, MW 386.31) (Le Guennec et al. Pediatrics, 76(5):834-40 (1985)). Caffeine citrate is indicated for the short-term treatment of apnea in premature infants between 28 and 33 weeks gestational age; it is considered safe for use in adults.

Other examples of vasodilatory agents include organic nitrates, such as nitroglycerin and amyl nitrate, glyceryl trinitrate, pentaerythritol tetranitrate, hydralazine, sildenafil citrate (e.g., VIAGRA, Pfizer, NY, N.Y.), minoxidil, diazoxide, sodium nitroprusside, isosorbide dinitrate, isosorbide mononitrate, cilostazol, papaverine, dipyridamole, oxyfedrine hydrochloride (HCl), diltiazem HCl, tolazoline HCl, hexobendine, bamethan sulfate, sulfonamide derivatives, such as dichlorphenamide, and combinations of vasodilatory agents.

Vasoconstrictors in accordance with the invention can include any compound capable of decreasing vascular blood flow in normal tissue. One example of a vasoconstrictor that can be used in accordance with the present invention is phenylephrine HCl. Phenylephrine HCl, or neosynephrine, is a powerful post-synaptic alpha-receptor stimulant with little effect on the beta receptors of the heart. It provides vasoconstriction that lasts longer than that of epinephrine and ephedrine, but its action on the heart contrasts with that of epinephrine and ephedrine as it slows the heart rate and increases stroke output while producing no disturbance in pulse rhythm. When administered by injection, phenylephrine is used to maintain adequate blood pressure and to treat certain types of irregular heartbeats (Chan, R. C. et al. JNCI, 72(1):145-150 (1984)).

Another example of a vasoconstrictor that can be used in a accordance with the present invention is pitressin. Pitressin, also known as vasopressin, causes contraction of smooth muscle of the gastrointestinal tract and of all parts of the vascular bed, especially the capillaries, small arterioles and venules with less effect on the smooth musculature of the large veins. It is most commonly prescribed as a diuretic (Dietz, D. et al. J. Surg. Res., 77:150-6 (1998)).

Other examples of vasoconstrictive agents which may be used in the present invention include pseudoephedrine, angiotensin, vasopressin, levonordefrin, epinephrine, naphazoline nitrate, tetrahydrozoline HCl, oxymetazoline HCl, tramazoline HCl, lypressin, and combinations of vasoconstrictive agents.

The vasoactive agents in accordance with the present invention can be administered to the patient by different routes. For example, the vasoactive agent can be administered intravenously, orally, rectally, by inhalation, transdermally, and peritoneally. It will be appreciated that the method by which the vasoactive agent is administered will depend on the specific vasoactive agent being used as well as the specific tissue in which the blood flow is being measured.

In an aspect of the invention, more than one vasoactive agent can be administered to the patient. For example, a first vasoactive agent can be administered to a subject followed by the administration of a second vasoactive agent a duration of time after administration of the first vasoactive agent. The second vasoactive agent can be the same as the first vasoactive agent or different than the first vasoactive agent. For instance, the first vasoactive agent administered to the subject can be a vasodilator, such as a 5-phophodiesterase inhibitor (e.g., silfenadil), and the second vasoactive agent can be a vasoconstrictor, such as pseudoephedrine.

The amount of vasoactive agent administered to a subject is that amount effective to noticeably (or perceptibly) modify (i.e., increase or decrease) the blood flow to and in the tissue of interest for a predetermined duration of time. The tissue of interest can include normal tissue surrounding the tissue of interest as well as normal and abnormal tissue in the tissue of interest. The amount of vasoactive agent administered can be determined by, for example, measuring increases or decreases in the blood volume per minute in vasculature (e.g., arteries) to the tissue of interest. In an aspect of the invention, the amount of vasoactive agent administered to the subject can be that amount effective to increase or decrease blood flow (blood volume per minute) in at least one artery to the tissue of interest by at least about 10%. In another aspect of the invention, the amount of vasoactive agent administered to the subject of interest can be that amount effect to increase or decrease blood flow (blood volume per minute) in at least one artery to the tissue of interest by at least about 15%.

The amount of vasoactive agent administered to the subject to cause a noticeable increase or decrease in blood flow to the tissue of interest will depend on the specific agent administered to the subject. By way of example, acetazolamide was administered to a subject intravenously at an amount of 100 mg/kg. Blood flow in the hepatic artery was found to have increased following administration of the acetazolamide from about 2.8 ml/min to about 3.1 ml/min after about 10 minutes and to about 3.3 ml/min after about 20 minutes. This correlates to an about 18% increase in the blood flow through the hepatic artery. In another example, caffeine citrate was administered intravenously to a subject at about 20 mg/kg and found to increase hepatic artery flow about 14%.

Following and/or during administration of the vasoactive agent, at 30, the rate of blood flow in the tissue of interest is determined to identify portions or areas of the tissue that show an increase or decrease in blood flow compared to surrounding areas of tissue. Normal tissue, following administration of a vasodilator, will show a marked increase blood flow. Abnormal tissue (e.g., neoplastic tissue) will show less blood flow compared to the normal tissue as the normal tissue can demonstrate a “steal” phenomenon as blood flow from the abnormal tissue is directed to the normal tissue. In contrast, following administration of a vasoconstrictor, normal tissue will show a marked decreased blood flow. Abnormal tissue (e.g., neoplastic tissue) will show greater or enhanced blood flow compared to the normal tissue. This difference in blood can be determined to distinguish normal tissue from abnormal tissue.

The rate of blood flow in the tissue of interest can be determined by generating one or more images of the tissue of interest using an imaging technique. Imaging techniques of the present invention can include, for example, X-ray-based imaging (i.e., radiography and computed tomography, CT), nuclear imaging, magnetic resonance imaging (MRI), and ultrasound.

Among the available imaging modalities, CT and MRI are widely recognized as the fundamental modalities for diagnosis, biopsy, local treatment, therapy planning, and post-procedure assessment of cancer. CT and MRI devices generate anatomic and functional information of organs by sampling tissues with different forms of energy and collecting unique diagnostic information related to molecular tissue parameters. CT scans are completed with the use of a 360-degree X-ray beam and computer production of images. These scans allow for cross-sectional views of body organs and tissues.

MRI is a diagnostic and research procedure that uses high magnetic fields and radio-frequency signals to produce images. MRI is based on the fact that the most abundant molecular species in biological tissues is water. In fact, it is the quantum mechanical “spin” of the water proton nuclei that ultimately gives rise to the signal in all imaging experiments. In MRI, the sample to be imaged is placed in a strong static magnetic field (1-12 Tesla) and the spins are excited with a pulse of radio frequency (RF) radiation to produce a net magnetization in the sample. Various magnetic field gradients and other RF pulses then act on the spins to, code spatial information into the recorded signals. In turn, MRI is able to generate structural information in three dimensions in relatively short time spans.

Ultrasound is another valuable diagnostic imaging technique for studying various regions of the body, including, for example, the vasculature, such as tissue microvasculature. Ultrasound involves the exposure of a patient to sound waves. Generally, the sound waves either dissipate due to absorption by body tissue, penetrate through the tissue, or reflect off of the tissue. The reflection of sound waves off of tissue, generally referred to as backscatter or reflectivity, forms the basis for developing an ultrasound image. In this connection, sound waves reflect differentially from different body tissues. This differential reflection is due to various factors, including the constituents and the density of the particular tissue being observed. The detection of the differentially reflected waves, generally with a transducer that can detect sound waves having a frequency of one megahertz (MHZ) to ten MHZ, provides the basis for detecting the waves and integrating them into an image which is quantitated and ultimately converted into an image of the tissue being studied.

To facilitate measuring the blood flow in the patient, it will be appreciated that a contrast agent can be administered to the patient. Contrast agents are chemicals used to enhance an image by increasing contrast between target and surrounding tissue(s).

For example, one particular aspect of CT, known as functional CT, takes advantage of the known pharmacokinetics of currently available contrast agents. When using CT imaging, a contrast material can be administered to a patient. The contrast material remains in the vascular blood pool for several minutes before it diffuses into the total body water pool. The precise local dynamics depend upon the local endothelial cell permeability, with variation arising between different organs and disease processes. Functional CT utilizes these concepts to gain quantitative perfusion information in a region of interest. A typical perfusion protocol collects repeated images of an area of interest before and during contrast bolus injection and clearance. A software package available from the scanner manufacture then fits data from aortic and venous time-density curves to the dual-input, single compartment model of liver perfusion.

Contrast agents useful for CT usually contain atoms, which are electron dense, such as bromine or iodine, and are efficient attenuators of X-ray radiation. By far the most common CT agents are monomeric or dimeric iodinated benzene rings with various pendent groups such as Oragrafin, Cholografin, and Renografin (Squibb Diagnostics, Princeton, N.J.). One important advance in the use of iodine-containing CT contrast agents has been the development of non-ionic contrast agents, such as the ones described by M. T. Kneller et al., PCT Application Ser. No. WO 93/10825 published 1993. Further examples of acceptable contrast agents for use with X-ray imaging include Optiray® (ioversol; Mallinckrodt Imaging) and Conray® (iothalamate meglumine; Mallinckrodt Imaging).

Functional MRI (fMRI), is the addition of contrast agents to provide a more detailed visual interpretation of anatomic structures. For both liver and breast evaluation, contrast agents add diagnostic information; however, improved contrast techniques are still needed to improve specificity.

Dynamic contrast enhanced MRI (DCE-MRI) is a perfusion imaging technique that has recently emerged as a promising method for imaging the physiology of the microcirculation and is based on the tracer kinetics of intravenous T1-shortening contrast agents. These kinetics are typically evaluated in terms of the rate of change of the tissue concentration and bi-directional transport of the tracer across the capillary endothelium between the plasma and the extravascular extracellular space (Tofts, P. S. et al. J. Magn. Reson. Imaging, 10(3):223-32 (1999); Hoffmann, U. et al. Magn. Reson. Med., 33(4):506-14 (1995); Port, R. E. et al. J. Magn. Reson. Imaging, 10(3):233-41 (1999); Knopp, M. V. et al. J. Magn. Reson. Imaging, 10(3):260-6 (1999); Lucht, R. et al. Magn. Reson. Med., 43(1):9-16 (2000); Henderson, E. et al. J. Magn. Reson. Imaging, 12(6):991-1003 (2000); Brix, G. et al. Eur. Radiol., 11(10):2058-70 (2001); Schnall, M. D. et al. Magn. Reson. Imaging Clin. N. Am. JID, 9(2):289-vi (2001)) and has recently been extended to investigations of anti-angiogenic (Brix, G. et al. Eur. Radiol., 11(10):2058-70 (2001); Schnall, M. D. et al. Magn. Reson. Imaging Clin. N. Am. JID, 9(2):289-vi (2001)) and vascular targeting tumor therapeutic agents (Dowlati, A. et al. Clin. Canc. Res., 7:2917-2976 (2001)). The latter entails predicting tumor response to planned therapy as well as tracking the effect of anti-angiogenic or vascular targeting agents on perfusion in vivo.

Contrast agents useful for MRI affect a change in the relaxation characteristics of protons which can result in image enhancement and improved soft-tissue differentiation. Different classes of MRI agents include positive and negative contrast agents. Positive contrast agents are typically small molecular weight compounds which have unpaired electron spins in their outer shells, such gadolinium, manganese, and iron. Examples of positive contrast agents include gadopentetate dimeglumine, gadoteridol, gadoterate meglumine, mangafodipir trisodium, gadodiamide, and gadoversetamide.

Negative contrast agents are typically small particulate aggregates, often termed superparamagnetic iron oxide, which produce predominately spin-spin relaxation effects. Additionally, there is a special group of negative contrast agents, known as perfluorocarbons, which are especially good MRI contrast agents because their presence excludes the hydrogen atoms responsible for the signal.

One particular class of contrast agents used with MRI, called gadolinium contrast compounds, function similarly to the iodinated agents used for CT. Depending upon vessel endothelial cell permeability, these compounds produce opacification of the blood pool for several minutes after injection and, subsequent to equilibration, opacifies the general body water space.

Gadolinium agents have become refined and are now widely used to provide more specific tumor vessel information. The most common method of contrast material assessment has been the study of signal intensity change over time intervals. The parameters often used are: peak enhancement; quantification of the initial and mean gradient upsweep of enhancement curves; maximum signal intensity; and, washout gradient (Gribbestad, I. S. et al. J. Magn. Reson. Imaging, 4(3):477-80 (1994)). Gadolinium agents have been commonly used for evaluation of liver tumors, either primary neoplastic, primary benign (focal nodular hyperplasia), or metastatic.

Contrast agents may also be used with ultrasound imaging. Exemplary contrast agents include suspensions of solid particles, emulsified liquid droplets, gas-filled bubbles, Levovist® (Schering AG) and Optison® (Mallinckrodt Imaging). See also, e.g., Hilmann et al., U.S. Pat. No. 4,466,442, and published International Patent Applications WO 92/17212 and WO 92/21382.

The images of tissue interest can be compared with a base line or reference image. The reference image can comprise, for example, an image of the tissue taken before or during administration of the vasoactive agent. Alternatively, the image can be compared to image of normal tissue, such as normal proximal the tissue of interest. By comparing intensity measurements between the reference image and subsequent image(s), a flow value (proportional to contrast agent change per area per time) can be calculated for the region of interest and the corresponding normal tissue.

In one aspect of the present invention, a first image of a tissue of interest can be generated by an imaging technique. A first image of normal tissue which corresponds to the region of interest may also be generated. A contrast agent may be administered to the patient prior to generating the first image. After administration of the contrast agent, a subsequent scan (or scans) may be performed of the area of interest. A subsequent scan (or scans) may also be performed on the corresponding normal tissue. By comparing intensity measurements between the first image and subsequent image(s), a flow value (proportional to contrast agent change per area per time) can be calculated for the region of interest and the corresponding normal tissue.

By evaluating the first and the subsequent images, the vascularity of the region of interest may be determined. More particularly, the vascularity of the region of interest may be determined by comparing the first and subsequent images with the images of the normal tissue. It may be determined, for instance, that the region of interest is hypovascularized (i.e., a vascular density less than the vascular density of the normal tissue). Alternatively, the region of interest may be hyperpolarized (i.e., a vascular density greater than the vascular density of the normal tissue).

Upon determining the vascularity of the region of interest, a particular vasoactive agent may then be selected. For instance, if the tissue of interest is hypovascularized, a vasodilatory agent may be administered. Alternatively, if the tissue of interest is hypervascularized, a vasoconstrictive agent may be administered. Further, a vasoactive agent may be administered without first determining the vascularity of the tissue of interest.

After selecting the vasoactive agent, the agent may be administered to the patient. A contrast agent may be administered prior to, or simultaneous with, the administration of the vasoactive agent. Thereafter, a second image of both the region of interest and the normal tissue may be generated. The first and second images may comprise parts of a single overall image sequence. A comparison of the overall image sequence for both the region of interest and normal tissue may then be made. Comparison of images from the tissue of interest and the normal tissue may identify any local variations in signal intensity resulting from changes in the rate of blood flow induced by the vasomodification.

Depending upon whether a vasodilatory agent or vasoconstrictive agent is administered, differences between normal and abnormal tissue may be detected. For instance, where a vasodilatory agent is administered, the rate of blood flow in a region of interest may decrease. A decrease in the rate of blood flow in a region of interest may indicate the presence of abnormal tissue. Alternatively, where a vasodilatory agent is administered, the rate of blood flow in a region of interest may not change. In this instance, the region of interest may comprise abnormal tissue. Further, administration of a vasodilatory agent to hypovascularized tissue may increase the imaging signal in normal tissue by increasing blood flow, and also increase the imaging signal in the region of interest due to a decrease in blood flow. These differences may provide better conspicuity of abnormal tissue.

Where a vasoconstrictor is administered, the rate of blood flow in a region of interest may increase. An increase in the rate of blood flow in a region of interest may indicate the presence of abnormal tissue. Further, where the tissue of interest is hypervascularized, a vasoconstrictive agent may be used to increase the imaging signal in normal blood flow (i.e., decrease blood flow) and to increase the imaging signal in abnormal tissue (i.e., increase blood flow). These differences may provide better conspicuity of abnormal tissue.

Another aspect of the present invention may include identification and classification of abnormal tissue. More particularly, the present invention may be used to determine whether abnormal tissue is either benign or malignant based on histological analysis.

A first image of a tissue of interest may be generated by an imaging technique. A first image of normal tissue which corresponds to the region of interest may also be generated. A contrast agent may be administered to the patient prior to generating the first image. After administration of the contrast agent, a subsequent scan (or scans) may be performed of the area of interest. A subsequent scan (or scans) may also be performed on the corresponding normal tissue. By comparing intensity measurements between the first image and subsequent image(s), a flow value (proportional to contrast agent change per area per time) can be calculated for the region of interest and the corresponding normal tissue.

By evaluating the first and the subsequent images, the vascularity of the region of interest may be determined. More particularly, the vascularity of the region of interest may be determined by comparing the first and subsequent images with the images of the normal tissue. It may be determined, for instance, that the region of interest is hypovascularized (i.e., a vascular density less than the vascular density of the normal tissue). Alternatively, the region of interest may be hyperpolarized (i.e., a vascular density greater than the vascular density of the normal tissue).

Upon determining the vascularity of the region of interest, a particular vasoactive agent may then be selected. For instance, if the tissue of interest is hypovascularized, a vasodilatory agent may be administered. Alternatively, if the tissue of interest is hypervascularized, a vasoconstrictive agent may be administered. Further, a vasoactive agent may be administered without first determining the vascularity of the tissue of interest.

After selecting the vasoactive agent, the agent may be administered to the patient. A contrast agent may be administered prior to, or simultaneous with, the administration of the vasoactive agent. Thereafter, a second image of both the region of interest and the normal tissue may be generated. The first and second images may comprise parts of a single overall image sequence. A comparison of the overall image sequence for both the region of interest and normal tissue may then be made. Comparison of images from the tissue of interest and the normal tissue may identify any local variations in signal intensity resulting from changes in the rate of blood flow induced by the vasomodification.

Depending upon whether a vasodilatory agent or vasoconstrictive agent is administered, differences between normal and abnormal tissue may be detected. For instance, where a vasodilatory agent is administered, the rate of blood flow in a region of interest may decrease. A decrease in the rate of blood flow in a region of interest may indicate the presence of abnormal tissue. Alternatively, where a vasodilatory agent is administered, the rate of blood flow in a region of interest may not change. In this instance, the region of interest may comprise abnormal tissue. Further, administration of a vasodilatory agent to hypovascularized tissue may increase the imaging signal in normal tissue by increasing blood flow, and also increase the imaging signal in the region of interest due to a decrease in blood flow. These differences may provide better conspicuity of abnormal tissue.

Where a vasoconstrictor is administered, the rate of blood flow in a region of interest may increase. An increase in the rate of blood flow in a region of interest may indicate the presence of abnormal tissue. Further, where the tissue of interest is hypervascularized, a vasoconstrictive agent may be used to increase the imaging signal in normal blood flow (i.e., decrease blood flow) and to increase the imaging signal in abnormal tissue (i.e., increase blood flow). These differences may provide better conspicuity of abnormal tissue.

Upon detection of abnormal tissue, a biopsy of the tissue may then be made. Excised tissue may then be subjected to histological and immunohistochemical analysis. As embodied in Examples 4 and 6, correlating the histological analysis of the excised tissue with the detected changes in the flow rate may permit classification of the excised tissue as either benign or malignant.

Yet another aspect of the present invention may include monitoring neoplastic tissue after radiofrequency ablation. RFA is typically used for treating tumors localized to certain organs such as the liver, kidney and adrenal glands. With this technique, relatively small probes are placed into the tumor and RF energy deposited. The RF energy causes the tissue around the tip of the probe to heat up to a high temperature above which cells break apart and die. Since RFA kills both tumor and non-tumor cells, the goal is to place the probes so that they destroy all of the tumor plus an adequate “rim” of non-tumorous tissue around it. Example 3 is illustrative of this aspect of the present invention.

The present invention is further illustrated by the following examples, which are not intended to be limiting.

### Example 1

### Functional CT Imaging of Tissue Perfusion During Tumor Development

The purpose of this study was to utilize perfusion CT to examine sequential vascular development of experimental hepatic tumors and quantify changes of arterial and venous contributions to total tumor and normal liver perfusion occurring with tumor growth. Within this scope, we examined the arterial and venous contributions to tumor perfusion and correlated tumor growth, in particular, enlargement of central necrosis, with the perfusion data. The resulting quantitative data contributes to the fundamental knowledge of liver tumor physiology and is directly applicable to future clinical tumor staging and therapy selection.

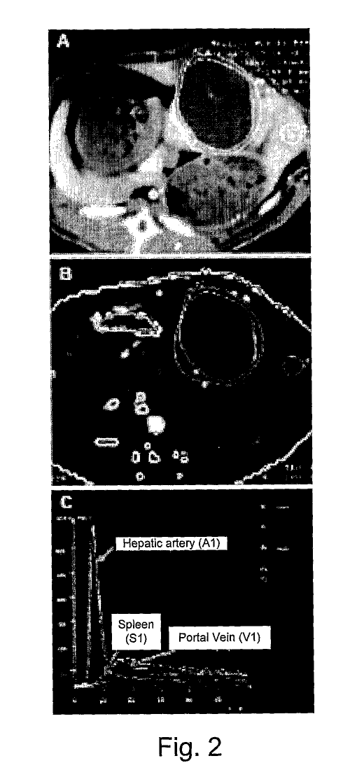

A VX2 tumor was inoculated in livers of 5 male New Zealand White rabbits (3 kg, Covance). The VX2 tumor, a transplantable hepatoma, was donated by S, Nahum Goldberg, MD, (Beth Israel Medical Center, Boston, Mass.). The tumor was first propagated in the thigh of a donor rabbit, where a suspension of tumor cells was injected intramuscularly into the thigh. The tumor was grown until the palpated size reached approximately 1 cm³ and was harvested from anesthetized animals. For implantation in the liver, frozen tumor was rapidly thawed and washed, and a piece was inserted into the liver parenchyma. The tumor was grown for 28 days and was examined with CT (Mx8000LDT, Philips Medical Systems, Andover, Mass.), on days 7, 14, 21 and 28 after implantation. During each exam, an initial, unenhanced baseline scan was carried out. Next, a 3 cc bolus of contrast (Conray®, Mallinckrodt Imaging) was administered through a 21-g catheter and placed in the central ear vein. The perfusion protocol, which imaged 8 consecutive slices every second for 1 minute, was executed using the following parameters: axial scan, 360° scan angle, 3 mm slice thickness, FOV 195 mm, 120 KV, 150 mAs/slice, and 0.5 sec rotation time. A CT perfusion software package available from the scanner manufacturer was used for all perfusion measurements. The software fits data from aortic and venous time-density curves to the dual-input, single compartment model of liver perfusion. This method has been described previously by a number of investigators (Kapanen, M. K. et al. Acad. Radial., 10(9):1021-1029 (2003); Dugdale, P. E. et al. Eur. J. Radial., 30(3):206-213 (1999); Miles, K. A. et al. Acad. Radial., 7:840-50 (2000)). The arterial and venous flow was measured in the viable peripheral rim of each tumor and in a portion of normal liver distant from the tumor implantation site. A representative CT perfusion protocol output is shown in FIG. 2. Total tumor size was determined from the CT scans by measuring the diameter of the largest tumor dimension (including the hypodense necrotic core and the enhanced outer rim) and calculating the area. The viable tumor rim area was calculated simply by subtraction of the necrotic core area from the total area. The tumor growth, necrosis data, and days of tumor growth were correlated with total, arterial and venous perfusion.

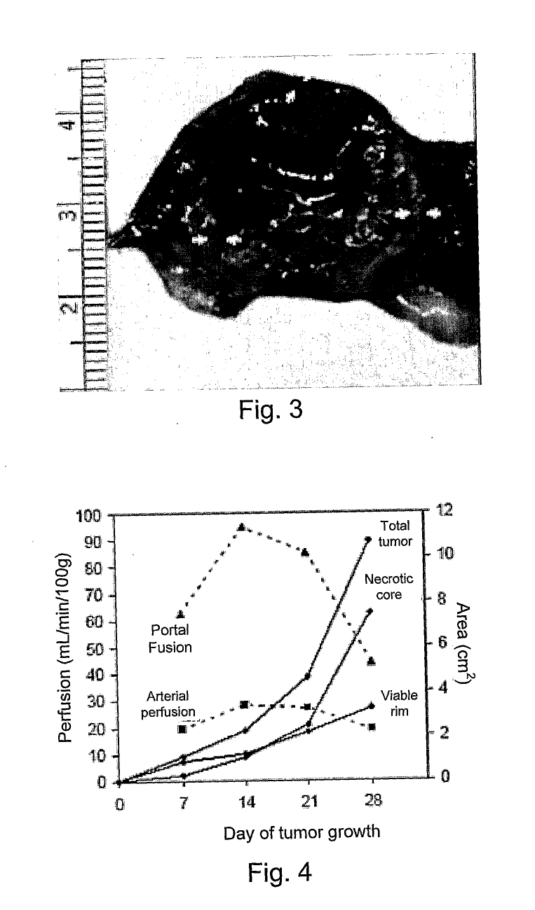

A typical VX2 tumor 28 days after implantation is shown in FIG. 3. Central necrosis occupies the majority of the tumor volume with the outer viable capsule having an approximate thickness of only 2 mm in a tumor diameter of over 2 cm. The tumor shown in FIG. 3 corresponds to the tumor CT images in FIG. 2. Quantitative analysis of the remaining CT perfusion data suggests that significant differences exists between perfusion of the viable tumor capsule and the surrounding normal liver throughout the duration of tumor development. The difference is most striking when examining the arterial perfusion, which is consistently greater in the growing tumor than in the normal liver, with statistically significant increases at days 7-21. The arterial perfusion in tumor increases sharply between days 7 and 14 (19±3 to 28±6 mL/min/100 g), remains stable until day 21, and then decreases markedly. Conversely, while the portal contribution to perfusion is identical in normal and tumor tissues for the first 21 days, it shows a drastic reduction between days 21 and 28 (85±21 to 44±19 mL/min/100 g), although this difference does not show statistical significance due to the high inter-animal variability.

The perfusion data can be correlated with the growth progression of the tumor as shown in FIG. 4. Furthermore, the parabolic pattern of arterial and portal tumor perfusion over the 28 day study duration can also be related to the pattern of tumor growth. The sharp increase in total tumor size and size of the central necrosis correspond to a drastic drop in portal perfusion and a slight decrease in arterial perfusion. Because the viable portion of the tumor increases at a constant rate throughout the study, one can also speculate that the initial blood supply to the tumor is primarily venous and advances towards an arterial one in the latter stages of tumor growth.

By determining the primary source of tumor blood supply and its progression with tumor development, the functional CT approach may be a useful tool in tumor staging, selection of optimal chemotherapeutic and anti-angiogenic agents, and their route of administration in patients. Temporal assessment of the changes in tumor vascularization aids in understanding such changes, and this understanding may translate into dynamic treatment regimens which intervene at appropriate times, i.e., angiostatin, alkylating agents, metabolic modulators, and genomic modifications.

### Example 2

### Vasoreactivity of Liver Tumors to Caffeine Citrate Measured by Perfusion CT

Caffeine citrate is generally used for treatment of apnea of prematurity in infants and for liver function tests. The effect of caffeine citrate on hepatic arterial blood flow is unclear due to both central and peripheral effects as well as dosage of this agent. Because caffeine citrate has minimal toxic effects on the liver, we tested the concept that caffeine citrate may cause vascular vasodilation in the hepatic artery and therefore may be useful in our approach to alter hepatic hemodynamics in normal and tumor tissue in the liver.

Using the same procedure as described for acetazolamide in Example 1, the effect of caffeine citrate on hepatic artery blood flow in 3 rats was examined. When caffeine citrate (20 mg/kg, IV bolus) was given, blood flow in the hepatic artery increased by 14% over the blood flow values prior to caffeine citrate as shown in Table 1.

TABLE 1
Hepatic Arterial Blood Flow Changes After Caffeine
Citrate Administration

| | | Caffeine | Percent | |
|---|---|---|---|---|
| | Control | Citrate | Change | P-value** |
| Flow (mL/min | 2.3 ± 0.7* | 2.67 ± 0.8 | +14% | 0.058 |
| Pressure (mmHg) | 123 ± 12 | 128 ± 13 | +4% | NS |
| Resistance (RU) | 70 ± 26 | 60 ± 20 | −14% | NS |

*all data in mean ± SE (n = 3)
**P-values calculated with paired, two-tailed, Student's t-test
RU—Resistance units = (mmHg/)/mL/min

Resistance in the hepatic artery fell while systemic pressure was not significantly affected. These changes suggest that caffeine citrate caused a direct vasodilation of the hepatic artery, which is consistent with the effects of other xanthines (i.e., methylxanthenes) (Kelleher, D. K. et al. Int. J. Radiat. Oncol. Biol. Phys., 42(4):861-4 (1998)).

### Example 3

### Vasoreactivity of Liver Tumors to Acetazolamide Measured by Perfusion CT

Despite remarkable advancements in clinical imaging technology, clear and unfailing methods of identifying malignant tissue from benign cystic lesions and normal tissue remain elusive. Currently accepted protocols range from manual selection of enhancing regions on CT and MRI scans, to injection of cumbersome radiotracers for nuclear imaging. All of these methods still require a follow up biopsy to identify the pathology of the tissue in question. In a related problem, follow-up imaging of procedures such as tumor radiofrequency ablation (RFA) also fails to demonstrate procedure success and remains dependent on tissue biopsy should uncertainty arise. The purpose of this preliminary study was to develop a straightforward, explicit method of distinguishing tumor from normal tissue on CT scans. The technique uses functional CT to measure perfusion in normal and tumor tissue before and after pharmacological manipulation of blood flow with a carbonic anhydrase inhibitor (CAI). CAI is a potent but localized vasodilator of normal blood vessels in tissues with high levels of carbonic anhydrase such as the liver, but should have no such effect on blood flow in malignant growths. Thus, the measured perfusion differences in response to the CAI can be used to differentiate tumor from normal tissue. The study is meant to demonstrate the potential for this technique in patient diagnosis and treatment follow-up, both of which are currently being addressed in ongoing studies.

### Materials and Methods

VX2 Tumor Model

All animal procedures were approved by the Institutional Animal Care and Use Committee at Case Western Reserve University. The VX2 tumor, a xenograft hepatoma model used frequently in radiological studies, was donated by S, Nahum Goldberg MD, (Beth Israel Medical Center, Boston, Mass.). Tumor was initiated in livers of 5 male New Zealand White rabbits weighing 3 kg (Covance, Princeton, N.J.) using methods described previously. The tumor was first propagated in the thigh of a donor rabbit, where a suspension of tumor cells was injected intramuscularly into one thigh. The tumor was grown until the palpated size reached approximately 1 cm³ and was harvested from anesthetized animals using sterile techniques. Pieces of the tumor (4 mm³) were cut, placed in 10% DMSO in pure calf's serum, and stored in liquid nitrogen.

For implantation in the liver, frozen tumor was rapidly thawed and washed. Rabbits were anesthetized using a combination of xylazine (5 mg/kg), ketamine (50 mg/kg), acepromazine (2 mg/kg) and atropine (0.2 mg/kg). The abdomen was shaved and cleaned, and the liver was exposed. A piece of tumor was then inserted into the liver parenchyma and the incisions were closed. During recovery, all animals received Buprenex (0.5 mg/kg) for pain management and a subcutaneous injection of 25 ml of 0.9% saline. Additional Buprenex and saline were given as needed.

### Functional CT Perfusion

Liver perfusion maps were created 28 days after initiation of the tumor by tracking contrast enhancement on eight sequential CT images (acquired on the Mx8000 IDT, Philips Medical scanner) before and after IV injection of acetazolamide. During each CT exam, first an unenhanced baseline scan was carried out. Next, a 3 cc bolus of contrast (Conray®, 282 mg/ml organically bound iodine; Mallinckrodt Imaging, Hazelwood, Mo.) was administered though a 21-g catheter placed in the central ear vein. The perfusion protocol imaged 8 consecutive slices every second for 1 minute and was executed using the following parameters: axial scan, 360° scan angle, 3 mm slice thickness, FOV 195 mm, 120 KV, 150 mAs/slice, and 0.5 sec rotation time. Next, 1.5 mL of CAI (Acetazolamide, 50 mg/kg, Bedford Laboratories, Bedford, Ohio) was injected into the ear vein slowly over 2 minutes and the perfusion protocol was repeated after a 5 minute delay.

A CT perfusion software package available from the scanner manufacturer was used for all perfusion measurements. The software fits data from aortic and venous time-density curves to the dual-input, single compartment model of liver perfusion. This method has been described previously by a number of investigators. In our analysis, regions of interest were manually drawn at the peripheral rim of untreated tumor and distal normal liver tissue before and after CAI injection. The general location of both measurements was consistent throughout the two consecutive scans. Arterial and portal perfusion were calculated by measuring the slope of the time-density curve before (arterial) and after (venous) peak splenic enhancement and normalizing by the vascular input. A mean percent change in perfusion before and after CAI injection was calculated. All data are reported in mean±SE.

### Results

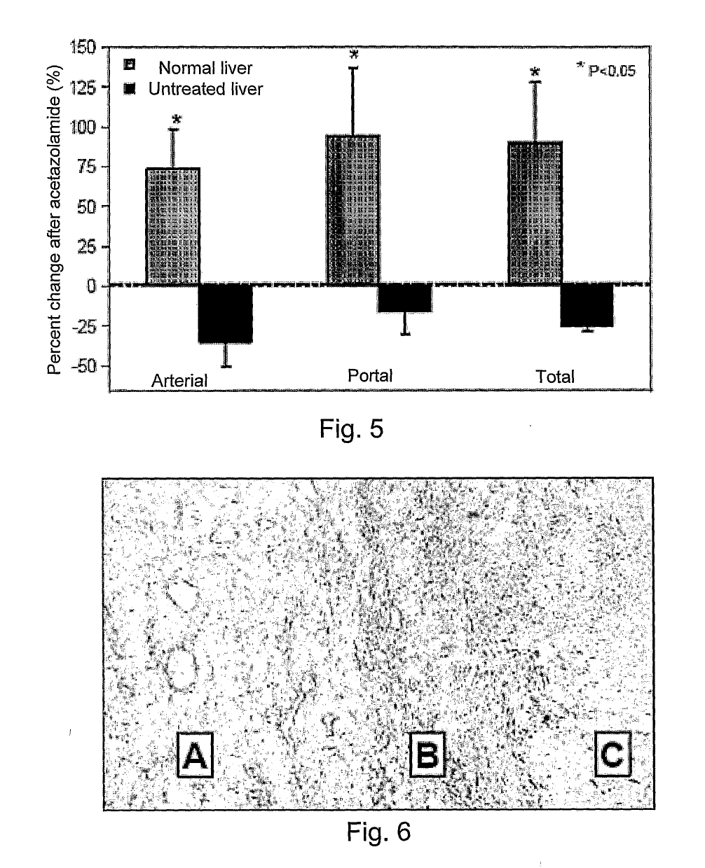

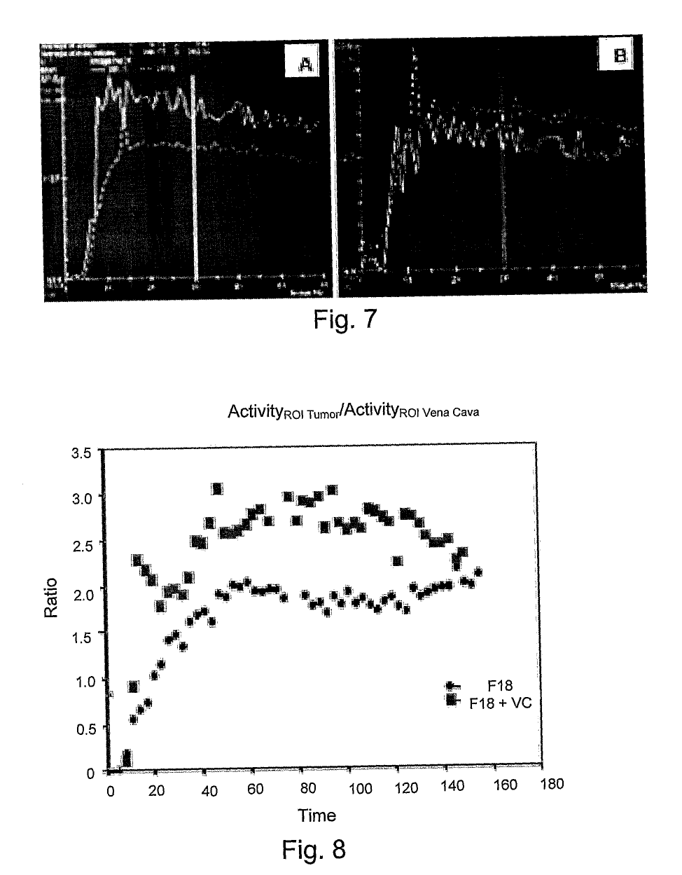

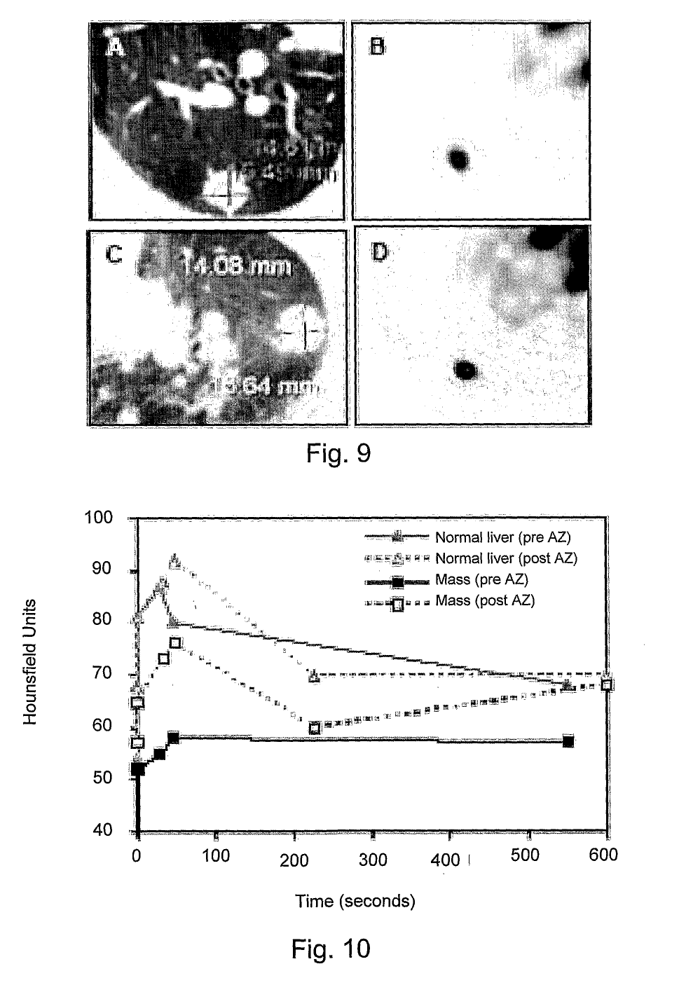

The peripheral rim perfusion of untreated tumors showed a marked decrease in arterial (−31±9%), venous (−20±12%) and total (−30±7%) perfusion after AZ administration. In the normal liver, arterial perfusion decreased negligibly (−3±33%), while venous (31±18%) and total (41±18%) perfusion increased after the AZ injection. Differences in normal and tumor flow were statistically significant for venous (p=0.04) and total (p=0.01) perfusion. From these changes, a simple equation of patient contrast: Patient contrast=100×(A−B)/A, where A is signal intensity of the patient and B is the signal intensity of the background, can be used to demonstrate “conspicuity” of the lesion. In simplest terms, conspicuity=normal tissue Hounsfield Units (HU)—lesion HU (Kuszyk, B. S. et al. Radiology, 217:477-86 (2000)), but contrast tends to be a more exact method of relating this data. This data is summarized in FIG. 5. The absence of vascular smooth muscle cells in the tumor was confirmed by histological analysis as shown in FIGS. 6(A-C).

### Discussion

Abnormalities in tumor vasculature have been studied extensively and are well documented. However, the application of the irregularities has not been sufficiently explored particularly in the imaging and interventional radiology fields. The current study uses multi-slice perfusion CT to visualize and quantify the perfusion in these abnormal tumors and compare their reaction to a potent vasodilator with the reaction of normal tissue. The striking differences in tumor response that were observed in this experiment can potentially be used to unequivocally distinguish malignant neoplasm from inflammatory response as part of an accurate post procedure assessment, and have the potential to outline the exact location of lesions for diagnosis and treatment planning.

In this ongoing study, functional CT was used to differentiate normal liver from an untreated experimental hepatoma after manipulation of blood flow with a vasodilator. We used multi-slice perfusion CT to visualize and quantify the perfusion in tumors and compare their reaction to a potent vasodilator with the reaction of normal tissue. The differences in tumor response that were observed can potentially be used to distinguish malignant neoplasm from inflammatory response as part of an accurate post procedure assessment, and have the potential to outline the exact location of lesions for diagnosis and treatment planning. The results demonstrate that CAI produces differential enhancement of tumors from normal liver. The end result is that lesions should be more conspicuous, facilitating detection. Future study will determine whether one can distinguish between malignant and non-malignant lesions by the same method.

### Example 4

### Characterization of Dose Response to Vasoactive Agents in the Liver

Various vasoactive agents have been administered to alter hepatic hemodynamics for many reasons including assessing liver function, increasing delivery of chemotherapeutic agents to tumors and improving detection of hepatic tumors. The aim of this experiment is to characterize some commonly used vasoactive agents; namely, acetazolamide, caffeine citrate, vasopressin, and phenylephrine. These agents have no toxic effects on the liver and negligible systemic side effects, and are routinely used. More particularly, the aim of this experiment is to characterize the effects of these agents on hepatic arterial blood flow and systemic blood pressure in a rodent model.

### Measurement of Alterations in Hepatic Artery Blood Flow in a Rabbit Model

Male New Zealand White rabbits (2-3 kg) are anesthetized with Inactin (BYK Guilden, Konstanz, Germany, 100 mg/kg), endotracheally intubated and placed on a heating pad to maintain body temperature. Inactin is used in these experiments because this drug has the least sympathetic stimulation and thus provides a stable time period for blood pressure measurements. Catheters are placed into the carotid artery for monitoring blood pressure via a recorder (Gould, Cleveland, Ohio) and into the external jugular vein for infusion of saline (0.6 ml/100 g body wt/hr) and vasoactive drugs. To measure blood flow in the hepatic artery, the hepatic artery is isolated through a midline incision, and an ultrasonic flow probe (Transonic, 0.5 VB 12, Ithaca, N.Y.) is placed across the common hepatic artery. Hepatic arterial blood flow is measured before and after the administration of the vasoactive drugs. Blood flow is measured in ml/min/100 g (body wt), and hepatic arterial vascular resistance is determined by dividing the systemic arterial pressure by the hepatic arterial blood flow and is reported as resistance units (RU). Differences in blood flow, blood pressure and hepatic arterial resistance between groups using different vasoactive agents is determined by One-Way Multivariate Analysis of Variance (ANOVA) with a level of significance set at 0.05 to determine differences among the groups.

### Experimental Protocol for Obtaining Dose-Response Curves

Using the model described above, dose response curves for acetazolamide, caffeine citrate, vasopressin and phenylephrine (Sigma, St. Louis, Mo.) are generated. Each drug is evaluated in 5 animals for a total of 20 animals. After placement of the ultrasonic flow probe on the hepatic artery, a baseline blood flow and pressure is recorded following a 15 minute stabilization period. Thereafter, one of the vasoactive agents is administered (in 0.2 mL total volume given over 2 minutes IV) starting at the doses as follows: acetazolamide, 5 mg/kg; caffeine citrate, 5 mg/kg; vasopressin 0.7 IU/kg; and phenylephrine, 0.02 mg/kg. These starting doses are based upon either literature values or our own values from preliminary experiments which show a minimal or small change in flow to the liver (Taki, K. et al. Angiology, 52(7):483-8 (2001); Le Guennec et al. Pediatrics, 76(5):834-40 (1985); Dietz, D. et al. J. Surg. Res., 77:150-6 (1998); Chan, R. C. et al. JNCI, 72(1):145-150 (1984)). Following the initial starting doses, hepatic arterial blood flow and systemic blood pressure responses are recorded for 15 minutes before the next dose is given. A total of 4 or 5 doses are possible depending on the blood pressure response. Normally, the subsequent doses is 2× the preceding dose and is adjusted as needed.

Differences in dose over time and changes in systemic blood pressure are analyzed by repeated measures ANOVA with a level of significance set at 0.05 to determine differences between groups. The use of 5 animals in five groups provides a statistical power of 0.8 assuming 5 repeated measures with a correlation of approximately 0.50 between measurements. The two optimal agents, one vasodilator and one vasoconstrictor, are selected based on the following criteria: 1) greatest effect on hepatic arterial flow with 2) the least effect on systemic blood pressure and cardiac output; and 3) shortest duration of induced change (i.e., an acute reaction). From the two selected agents, the lowest effective dose (LED) is used in all subsequent trials. Here, we define LED as that which yields a net change in hepatic blood flow of at least 15%, or three standard deviations above the mean.

### Correlation of Flow-Probe Measurements and CT and MRI Perfusion Measurements

Upon selection of the two best agents and their respective optimal doses, the normal liver blood perfusion in rats is measured with CT and MRI (as described in Example 5). The dose response study is carried out as above, but without the flow meter. The animals undergo a baseline perfusion scan, and the same can is repeated after administration of the vasoactive drug at the optimal dose. The animal recovers for 15 minutes and an additional dose of the drug is administered. Then, once again, the perfusion is measured with imaging. The imaging of perfusion data is correlated with the flow probe to confirm that the changes in perfusion are detected with CT and MRI.

### Example 5

### Functional MR and CT Imaging of Experimental Liver Tumors with and without Vasoactive Agents

This experiment addresses the need for an optimized diagnosis and follow-up method after radiofrequency ablation (RFA) of neoplasms. Through direct pharmacological manipulation and non-invasive imaging of blood flow in normal tissue, tumor tissue, and ablated tissue undergoing repair, a screening test is provided. More particularly, this experiment investigates this approach in animal models, utilizes progressive imaging methods, and validates the results with histological examination of excised tissue.

### Optimization of VX2 Tumor Model

The VX2 tumor is introduced into the livers of 10 male New Zealand White rabbits. Before each experiment, the tumor is first propagated in the thigh of another rabbit. Frozen VX2 cells are thawed, washed and re-suspended in HBSS. The suspension is injected into a donor rabbit thigh, and the tumor matures until the size reaches 0.5 cm (7-14 days). Progress is monitored with ultrasound (US). During each US exam, the tumor size is measured. On the day of surgery, a donor animal is euthanized and the thigh tumor is isolated and sectioned into 1-mm³ pieces.

### Treatment Groups

For inoculation of the tumor in a liver, a small section of tumor is inserted into the liver of four anesthetized rabbits (3-3.5 kg) using a trocar needle under aseptic conditions. Anesthesia is established with an isoflurane gas system (EZ-Anesthesia™, Euthanex Corporation). The system delivers a precisely blended mixture of oxygen and isoflurane through a lid unit that covers a host cage, and subsequently through a nose cone on the operating table. Buprenex (0.03 mg/kg) and 30 cc of 0.9% saline (SQ) is given prior to recovery. At this point, the tumor is treated with percutaneous RFA under image guidance, or left untreated. The treatment groups are shown in Table 2.

TABLE 2
Experimental Animal Groups

| | | | Group | |
|---|---|---|---|---|
| Group | Experiment | Model | Size | Endpoint |
| 0 | Tumor Donor | VX2 | n = 5 | Day 7-14 after |
| | | tumor/thigh | | tumor injection |
| 1 | VX2 tumor, no treatment | VX2 | n = 10 | Day 28 after |
| | | tumor/liver | | tumor injection |
| 2 | VX tumor, RFA treatment | VX2 | n = 10 | Day 28 after RFA |
| | | tumor/liver | | |
| 3 | Normal liver tissue (no tumor); | No tumor | n = 5 | Day 28 after RFA |
| | RFA treatment | | | |

As noted in Table 2, ablation is also carried out on animals with normal liver (Group 3), which is used to assess the perfusion in normal would healing response to ablation injury. The procedure begins with a baseline US scan to determine the location of treatment and to guide the insertion of the radiofrequency needle electrode into the approximate center of the tumor, or normal liver lobe, through a small incision in the abdomen. The tissue is ablated for 7 minutes at 90° C. The time is increased from the 5 minutes used previously. The animals are allowed to survive for 28 days. If any discomfort is perceived, the animals are euthanized at an earlier time. Differences in tumor diameter and volume are also measured on the images. Repeated ANOVA is utilized to determine differences between groups. The probability level of significance is established at 0.05.

### Multi-Modality Perfusion Measurement

Tumor assessment takes place at 7, 14, 21 and 28 days after implantation and immediately after the RFA procedure. Additional scans take place at 7 and 14 days after ablation. During each session, an anatomical CT, perfusion CT, and perfusion MR scan is done on anesthetized animals before and after vasoactive agent injection. The scans are performed with the Philips MX-8000 IDT™ CT, and with a Siemens 1.5T MRI scanner, when available (in the animal experiments, the bulk of experiments are carried out with CT). Five animals receive a vasodilator and 5 receive a vasoconstrictor in both the untreated and RFA groups.

### Functional Perfusion CT Data Collection and Analysis

A typical CT perfusion study consists of repeated scans of a selected area (e.g., a liver tumor) in which the perfusion is measured. First, a baseline scan of the area is acquired. Next, a bolus contrast injection (3 cc at 1.5 cc/sec) is administered, followed by scans of the area at 1-second intervals until all contrast has been washed out of the area. The perfusion protocol images 8 consecutive slices every second for 1 minute with the following parameters: axial scan, 360° scan angle, 3 nun slice thickness, FOV 195 mm, 120 KV, 150 mAs/slice, and 0.5 sec rotation time.

Through subsequent intensity measurements and collection of data for the entire scan time, a flow value (proportional to contrast change per area per time) is calculated for selected regions of interest. From each perfusion scan, the arterial and venous perfusion in the boundary region of ablated necrotic tissue and viable liver is calculated. The mean and standard deviation of measurements in three separate areas is calculated. Normal liver perfusion is determined in a similar manner. Tumor diameter and volume is measured on the images. Student's t-test is utilized to determine statistically significant differences in blood flow before and after vasoactive agent administration. The results, which are correlated with subsequent histology studies, serve to provide a means of assessing the completeness of the therapy when combined with the vasoactive injection.

A second method of CT data evaluation is used to correlate with recent perfusion. At different time intervals following contrast injection, pre- and post-vasoactive drug region of interest (ROI) measurements of Hounsfield numbers or signal intensity are made over the tumor, normal tissue, aorta, vena cava, and portal vein as the basis for a time flow analysis. The data is used to determine the conspicuity as the differential between the normal and abnormal tissue. Mathematical modeling is used to correlate the consistency of the time contrast curve between the pre- and post-vasoactive agent administration.

### DCE-MRI Data Acquisition and Analysis

Perfusion MRI studies commence with localization of the tumor or ablated tissue on preliminary scout scans. A representative slice of the pathology being evaluated is selected and, at this location, two spin-echo sequences with different repetition times (TR=400 or 800 msec, TE=15 msec, FA=90°, NA=2, FOV=200×200 mm, matrix=256×256) are acquired for the purpose of calculating pre-contrast T1 values. At the same slice location, the DCE-MRI is subsequently performed, and consists of 80-100 T1-weighted FLASH images (TR=37 msec, TE=4 msec, FA=30°, NA=1, FOV=200×200 mm, matrix=256×256) obtained prior, during, and after a 30 sec IV infusion of a 0.2 mL/kg (0.1 mmol/kg) dose of gadolinium-DTPA, followed by a 30 sec saline flush performed using an automatic power injector (Spectris, MEDRAD, Indianola, Pa.).

In this experiment, a DCE method is used similar to that outlined in the consensus recommendation for DCE-MRI presented at the 8^{th} annual meeting of the International Society for Magnetic Resonance Medicine (Evelhoch, J. L. et al. ISMRM (2000)). In summary, pre-contrast T1 maps are calculated numerically using the dual spin-echo technique. Raw DCE-MRI images are converted to tracer concentration maps using the signal expression for FLASH. The relationship between Gd-DTPA tissue concentration and T1 values is modeled using the fast water exchange approximation. An expression for the relative signal increase with respect to pre-contrast signal is then derived from these data. Prior to processing, FLASH and spin echo images are inspected for in-plane registration, and corrective shifts are applied to improve registration where necessary.

### Statistical Data Analysis

Absolute perfusion measurements or signal intensity measurement in normal tissue and tumor ROIs before and after vasoactive agent administration are collected from each experiment. For the purpose of this analysis, it is assumed that the VX2 tumor is malignant. From this data, the net percent change in flow is calculated for each ROI and each agent. In addition to ANOVA, the data is then placed in a contingency table and the Chi-squared statistic used to analyze the contingency table. A probability of less than 0.05 shows a significant relationship between a change in perfusion and the tissue type. This translates to a Chi-squared value greater than 5.991 based on a table with 2 rows and 3 columns translating to 2 degrees of freedom at a 95% confidence interval.

In summary, there are two different ways in which this method can be used to improve detection and classification of the lesions. In the simplest sense, if the relative perfusion measurements are examined and compared before and after VA administration, a negative correlation would suggest that a lesion is neoplastic. For example, if a vasodilator is administered, the healthy tissue perfusion should increase while a neoplastic lesion perfusion should decrease or remain the same. The converse should be true if a vasoconstrictor is used. In contrast, if a direct increase in subject contrast is desired to enhance the detection of the lesion, one first needs too ascertain if the lesion in question is hyper- or hypo-vascularized based on baseline contrast enhanced scans, and then the appropriate agent needs to be selected. For example, if the lesion in hypervascularized, a vasoconstrictor would be used to lower the signal in normal tissue (i.e., decrease blood flow) and to increase the signal in the lesion (i.e., increase blood flow), thereby increasing conspicuity.

### Example 6

### Correlation of Net Perfusion Change in Response to Vasoactive Agent with Histological Evaluation of Tissue

In order to understand the underlying processes behind the responses of tumor and normal tissue to the vasoactive agents, it is critical to carry out histological analysis. By correlating net perfusion changes in response to vasoactive agents with histological evaluation of tissue, analysis of the four different possible mechanisms responsible for visualized perfusion changes is possible. Methods used to analyze the possible mechanisms include: 1) correlating general contractility with the presence of smooth muscle cell specific actin (SMSA); 2) correlating responsiveness to acetazolamide with the presence of carbonic anhydrase IX (CA-IX); 3) evaluating the level of hypoxia by measuring the presence of lactate dehydrogenase-5 (LDH-5) (which plays a role in the anaerobic cellular metabolism by catalyzing the transformation of pyruvate to lactate); and 4) determining the presence of microvessels/endothelium in tumor vessels as marked by anti-positive for anti-factor VIII-antigen staining (Nasu, R. et al. Br. J. Cancer, 79(5-6):780-6 (1999)). Tumors treated with radiofrequency ablation (RFA) are subjected to additional analysis that includes measurement of the ablated region and staining for live versus dead cells.

### Histological Analysis for All Treatment Groups

For immunohistochemical staining of tissues for CA-IX, LDH-5, fVIII antigen, and SMSA, retrieved tissues are either frozen in liquid nitrogen on blocks containing OCT embedding medium, or fixed in 10% buffered formalin. Frozen sections are cut at 3-5 μm and frozen at −70° C. until staining. Formalin fixed tissue is embedded in paraffin and sections cut at 3 μm in onto charged “Plus” slides and dried in a 60° C. oven for 1 hour. The general procedures for immunostaining are described by (Wood, L. S. et al. Horiz. In Cancer Therap., 3:24-25 (2002)) in detail for the formalin fixed paraffin embedded sections. Briefly, slides are deparaffinized, rehydrated and placed in a 3% H₂O₂/H₂O bath for 10 minutes to quench endogenous hydrogen peroxide within the tissue. After a water rinse, slides are subjected to high heat epitope enhancement in 10 mM Citrate Buffer, pH 6.0. After a brief cooling, slides are rinsed in water and either placed on the Dako Autoimmunostainer, or placed in a humidity chamber for overnight incubation of primary antibody then finished on the immunostainer. All primary polyclonal and/or monoclonal antibodies are commercially available (DAKO Corporation, Santa Cruz Biotechnology Inc., or Rockland Immunochemicals). Optimum dilutions of all antibodies on selected tissue sections, as well as positive control tissues, is determined. Detection is achieved using standard horseradish peroxidase (HRP) labeled streptavidin-biotin (LSAB2; Dako) technology with 3-3-diaminobenzidine as the chromagen, resulting in a brown/black color change at the sites of antigen deposition. Slides are counterstained with hematoxylin, dehydrated and coverslipped. For image analysis and quantification of the staining reaction, digital imaging techniques developed by Dr. Ziats and colleagues (Wood, L. S. et al. Horiz. In Cancer Therap., 3:24-25 (2002)) are used. Digitized images from the slides are captured using a video microscopy system consisting of a light microscope (BX60, Olympus), video camera (DXC-390, Sony), coupler (U-TV0.35XC, Olympus), position encoded motorized stage (ProScan, Prior Scientific, Rockland, Mass.), and software (Image-Pro with Scope-Pro, Media Cybernetics). Background noise is removed from digital images and further processing of the images is accomplished using MATLAB. Each image is separated into 200×200 pixel (i.e., 0.4×0.4 mm) sub-images to provide sufficient information to constitute a “data point.” The color images are converted from RGB format into HSV format for color segmentation and the expression strength or density of the antibodies is calculated by relative number of brown pixels (fractional area) in an image.

### TUNEL Assay for Detection of Apoptosis

Apoptosis of endothelial cells and tumor cells is determined by a standard assay, the DNA fragmentation or TUNEL assay (Wassberg, E. et al. Am. J. Pathol., 154:395-403 (1999)). After deparaffination, sections are digested by proteinase K (20 μg/mL) for fifteen minutes. After rinsing in water, the sections are blocked in PBS solution containing 2% hydrogen peroxidase. A commercially available apoptosis kit, Apotag (Oncor, Gaithersburg, Md.) is used according to the manufacture's directions. As a positive control for some sections, DNase I is added after blocking in hydrogen peroxidase, producing DNA breaks in cells. Terminal deoxynucleotidyl transferase replaced with water serves as the negative control. The slides are counterstained with hematoxylin and positive cells per high power field (400×), and sections are quantitated by counting the number of apoptotic cells/high power field.

Histological Analysis and Correlation with Image Data for RFA Treated Animals

In addition to the processing mentioned above, a separate correlation is carried out for the tumors treated with RFA. At the completion of the experiment, animals are euthanized and their livers excised. The tissue is then cut parallel to the needle track in the center of the lesion, and the visible coagulative necrosis region is measured immediately with calipers in fresh tissue before fixation. A portion of the tissue is soaked for 40 minutes in 2% 2,3,4-triphenyltetrazolium chloride (TTC, Sigma Aldrich) solution. TTC, a marker for mitchondrial enzyme activity, is used to distinguish viable tumor cells from other cellular material as previously described by (Goldberg, S, N. et al. Radiology, 228(2):335-45 (2003)). All tissue is fixed in 10% formalin, embedded in paraffin, and sectioned to 5 μm. H&E and Masson's trichrome stains are used. Staining with Masson's trichrome reveals arterial collagen and confirms the presence of smooth muscle cells.

### Example 7

### Evaluation of Vasoactive Agents in Liver, Kidney and Breast Cancer Models

Correlation of Biopsy Findings with Changes in Tissue Perfusion in Humans

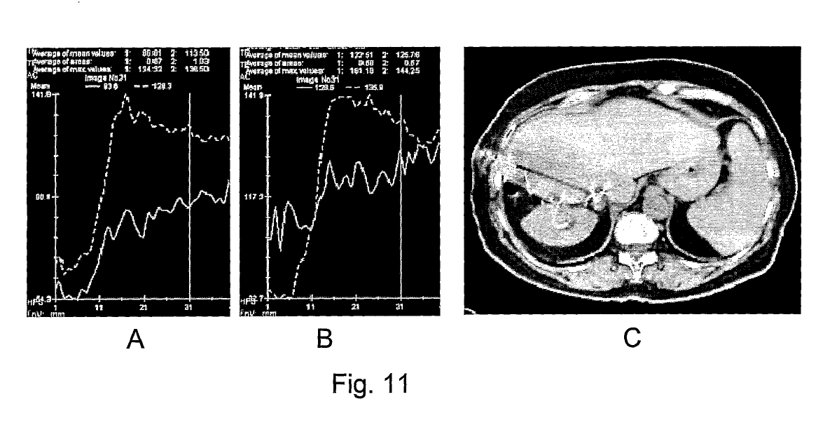

Several preliminary studies have been carried out using a protocol approved by the Institutional Review Board (IRB) of University Hospitals of Cleveland (FIGS. 10-11). One patient undergoing routine biopsy was screened using a pre- and post-acetazolamide injection contrast-enhanced CT scans. Following the scans, a biopsy of the tissue was taken and sent for routine histological analysis. On the image data, standard region of interest (ROI) analysis was used to obtain signal intensity values (in Hounsfield units or HU) over 600 seconds in normal liver and the mass. Imaging results are shown in FIG. 10 for a benign lesion. It is apparent, that the desired responses was achieved in this case, as blood flow, and thus signal intensity, increases in the normal liver and in the mass. This data was collected without benefit of dosage/kinetic study or optimization of technique. Our general hypothesis is that normal vessels either in normal liver or benign lesions respond to the vasodilatation, so the decrease in conspicuity results from dilatation of both vessel sites. Based on this hypothesis, the decrease in conspicuity from 32 to 15 HU after AZ injection suggests that the lesion is indeed benign. This was confirmed by histological diagnosis. A patient with a suspected recurrent tumor underwent a similar protocol as above. Again, on the baseline scan, the lesion enhances less than the normal liver. On the post Diamox (vasodilator) scan the enhancement of the lesion increases relative to the normal liver rather than decreases as one would expect from a malignant lesion. Instead of creating a “steal”, the flow increases flow in the lesion indicating it has normal vessels consistent with benign inflammation. Again, the results were confirmed with CT-guided biopsy.

### EXPERIMENTAL

The purpose of this experiment is to determine the applicability of combined vasoactive agent administration and imaging methods to different tissue environments other than the liver and models that closely resemble human cancers. Briefly, perfusion scans with and without vasoactive agent enhancement are repeated on tumors inoculated into the kidneys and breast of New Zealand White (NZW) rabbits and on woodchucks bearing naturally occurring hepatocellular carcinomas. The vasoactive is selected based on the nature of the lesion (i.e., whether it is hyper- or hypo-vascularized). The tumor development is tracked over time, and the imaging results are correlated with the tumor type as determined by histological and immunohistochemical markers.

Tumors are initiated in female NZW rabbit mammary fat tissue (n=10) and male NZW rabbit kidneys (n=10). The procedure for propagation, donation, and implantation of the tumor is identical to that in Example 1. The tumor growth is monitored with CT and MR perfusion at days 7, 14, 21 and 28, and the enhancement of conspicuity is measured as in Example 5.

### VX2 Rabbit Breast Tumor Model

Bilateral tumors are inoculated in the mammary pad of 10 female NZW rabbits using a procedure similar to that described in Example 5 and literature. Briefly, the tumor is initially grown in the thigh of a donor rabbit by injection of a previously frozen cell suspension. The tumor is grown for 710 days, or until a nodule can be palpated. On the donation day, the donor animal is euthanzied and the tumor is divided into four small sections (1×1 mm). These are implanted into the mammary pads of two rabbits under aseptic conditions and isoflurane anesthesia. To carry out the implantation, a small incision is made in the implantation site, and the tumor tissue is embedded in place. Gelfoam along with an absorbable suture are used to seal the implantation site.

### VX2 Tumor Model in the Kidney

Unilateral VX2 tumors are inoculated in the kidneys of 10 NZW rabbits using a procedure similar to that in Example 5 and literature (Horkan, C. et al. J. Vasc. Interv. Radiol., 15(3):269-74 (2004); Lee, J. M. et al. Eur. Radiol., 13(6):1324-32 (2003); Imai, S. et al. Acta. Radiol., 30(5):535-9 (1989)). On the donation day, the donor animal is euthanized and the tumor divided into two small sections (1×1 mm). These are implanted into one kidney per rabbit under aseptic conditions and isoflurane anesthesia. To carry out the implantation, the abdomen of each rabbit is opened with a midline incision. The kidney is exposed, and a small incision is made in the kidney capsule, making sure that the kidney itself is not perturbed. The tumor tissue is placed directly under the capsule and sealed in place with Gelfoam and an absorbable suture. The abdomen is closed in layers.

### Woodchuck Hepatitis Virus Infection and Hepatocellular Carcinoma

Woodchucks are obtained from the breeding facility at Cornell University inoculated with early stage hepatoma. After their arrival the Animal Resource Center at Case Western Reserve University, the woodchucks are acclimated for 1 week. On the day of the study, an animal weighing approximately 4 kg is initially anesthetized via intramuscular injection of a mixture of 5 mg/kg xylazine and 50 mg/kg ketamine. An endotracheal tube is inserted, and anesthesia is maintained using approximately 1.25% isoflurane adjusted to effect. Once anesthetized, the animal undergoes the CT and/or MRI imaging protocol described in Example 5. The scan protocol is repeated weekly for 4 weeks, and the animal is sacrificed at the conclusion of the time period. The liver is then harvested, and the tissue undergoes histological evaluation as described in Example 6. The total number of woodchucks is 10 with equal number used with the selected vasoconstrictor and with a vasodilator.

### Monitoring of Tumor Perfusion

CT and MRI perfusion measurements are carried out under isoflurane anesthesia using an identical protocol to that described in Examples 4 and 5. To summarize, tumor perfusion assessment takes place at 7, 14, 21 and 28 days after tumor implantation. During each session, an anatomical CT and perfusion CT scan is done on anesthetized animals before and after vasoactive agent injection. Five animals receive a vasodilator and 5 receive a vasoconstrictor. In a typical CT perfusion study, repeated scans of a selected area are acquired before and after vasoactive agent injection. A flow value (proportional to contrast change per area per time) is then calculated for selected regions of interest. From each perfusion scan, the perfusion in the normal tissue and tumor is measured. The net change in perfusion is correlated using a contingency table and the Chi-square statistic to the histological study results. Histological and immunohistochemistry of the excised tumors are identical to that described in Example 6.

### Example 8

### Correlation Vasoenhanced Imaging Diagnosis Results to Lesion Type in Humans

In this study, patients are evaluated for the use of acetazolamide in the CT perfusion technique. Consenting patients undergoing routine image-guided biopsy for diagnosis of unknown lesions receive a baseline contrast enhanced scan and a secondary scan following injection of one of the selected vasoactive agents. Histological analysis is used to identify whether the lesion is malignant or benign and the results correlated with the net change in blood flow to the lesion. More particularly, to evaluate the post-contrast attenuation characteristics of lesions referred for CT guided percutaneous biopsy, three study groups are established: 1) percutaneous biopsy group; 2) explanted neoplastic/cirrhotic liver associated with liver transplant; and 3) patients referred for MRI of the breast.

### Group 1—Percutaneous Biopsy

All patients referred for image guided percutaneous biopsy (n=200; 100 adult male and 100 adult female) are given the opportunity to enroll in the vasoactive study group. Patients who consent undergo the standard explanation of risks, benefits, and alternatives associated with the procedure, including the injection of intravenous contrast material. Additional information regarding optional enrollment into the study and the chosen vasoactive drug are also provided. All patients agreeing to participate do so by informed written consent.

The first step for all patients undergoing CT guided biopsy, in the absence of contraindication to contrast administration, is an enhanced pre-procedure localization scan through the area of interest to evaluate general vascularity. These planning scans are focused and limited, consisting of only 5-10 images (while a routine diagnostic liver CT may contain 150-300 images). Further, the standard volume of contrast given for a pre-procedure scan is halved for each scan so that the total volume of contrast administered is unchanged from normal clinical routine. Enrolled patients in the study receive 250 mg of acetazolamide or other vasoactive agent intravenously following the usual scan. The patient's cardio-respiratory status is continuously monitored. Five minutes following the acetazolamide injection, the second bolus enhanced scans are obtained. The patient then resumes the normal biopsy process. The vital signs and clinical status of all patients undergoing CT guided biopsies are monitored in the outpatient surgery center for 3-6 hours following the procedure. The specific deviations from normal clinical routine that the study participants undergo are: 1) the administration of acetazolamide; and 2) an additional contrast enhanced localization scan.

The following exclusion criteria are applied to ensure patient safety: a history of hypersensitivity to acetazolamide or sulfa drugs, pregnancy, and/or maintenance acetazolamide therapy. Sparse anecdotal literature exists to support cross-reactivity of the acetazolamide sulfa moiety; and acetazolamide has been shown to cross the placenta in animals. In addition, patients receiving maintenance acetazolamide therapy for any medical condition are excluded in order to avoid disrupting an established clinical equilibrium. Patients are routinely screened for current medications, drug allergies, and pregnancy, as well as for medical conditions associated with tenuous fluid balance (because of contrast volume) prior to undergoing a CT guided intervention. Otherwise, all adult patients referred for CT guided percutaneous biopsy are given the opportunity to participate in the study.

Explanted Neoplastic/Cirrhotic Liver Associated with Transplant

The second patient group studied are those individuals who have their liver explanted in anticipation of liver transplant. These patients have a generalized surveillance scan of the liver periodically during their transplant waiting period, which includes a standard enhanced and a second vasoactive enhanced CT scan. Following removal of the liver, the organ is sectioned at 8-10 mm thicknesses by a hepatopathologist. These sections are correlated with the appropriate axial scans and samples taken from local lesions for definite histopathology. These specimens are studied with standard stains, but also the specialized vascular stains (i.e., CA-IX, LDH-5, fVIII, SMSA).

### Patients Referred for MRI of the Breast

The third patient group is those patients referred for MRI of the breast. Patients who are having an MRI for evaluation of breast masses are given the opportunity to enroll in a vasoactive study related to breast mass characterization. These patients receive the routine MRI scan of the breast using gadolinium. Following administration of the selected vasoactive agent, a limited flow study is performed over any detected masses to determine if vasoactive changes occur. The study is done on a Siemens 1.5T MRI using a breast coil. A variety of pulsing sequences are used to detect any suspicious nodules, and a series of sequences encoded to that site are used to collect data during the intravenous injection of gadolinium, as described by Kuhl et al. (Kuhl, C. et al. Radiology, 211:101-11 (1999)) and Buadu et al. (Buadu, I. et al. Radiology, 200:639-49 (1996)). Parameters evaluated include time intensity profiles, curve analysis, maximum intensity values, and inflow and outflow gradient rates.

### Data Analysis

Absolute perfusion measurements or signal intensity measurement in normal tissue and lesion regions of interest (ROI) before and after vasoactive agent administration are collected from each experiment. From these data, the net percent change in flow is calculated for each ROT and each agent. Following conclusive diagnosis of the tissue based on histology, the data is placed in a 3×3 contingency table with 4 degrees of freedom. The Chi-squared statistic is used to analyze the contingency table. A probability of less than 0.05 indicates a significant relationship between a change in perfusion and the tissue type. This translates to a Chi-squared value greater than 9.441 based on a table with 3 rows and 3 columns translating 4 degrees of freedom at a 95% confidence interval.

### Histological/Immunohistochemical Staining of Biopsy Specimens

Immunohistochemical staining of tissue is performed with the peroxidase-antiperoxidase staining method for paraffin-embedded or frozen sectioned tissue as described in Example 6. Antibodies used for identification of endothelium include factor VIII antigen/von Willebrand factor (fVIII/vWF, rabbit antihuman polyclonal antibody, 1:3000, Dako Corp.), CD31 (PECAM-1, mouse monoclonal antibody, 1:25, Dako Corp.), and CD34 (mouse monoclonal antibody, 1:50, Coulter-Immunotech). fVIII staining is used for identification of endothelium of blood vessels. CD31 and/or CD34 monoclonal antibodies (as well as fVIII monoclonal antibodies) are considered if staining is less than optimal. For identification of smooth muscle cells (or pericytes), antibody to smooth muscle cell actin (SMSA, mouse monoclonal, 1:50, Dako Corp.) is used and is shown in FIGS. 5(A-C). The usefulness of identifying smooth muscle cells is to distinguish mature (positive smooth muscle) from immature (absent smooth muscle) vessels (Darland, D. C. et al. J. Clin. Invest., 103:157-8 (1999); Hlatky, L. et al. J. Natl. Cancer Inst., 94:883-893 (2002)). Other stains used include H&E for cellularity, Masson's trichrome for collagen and noncollagen proteins, and Ki67 for mitotic index (Prall, F. et al. Histopathology, 42:482-91 (2003)). Slides are observed for a brown reaction product and stopped by immersion in water and then counterstained with hematoxylin, cleared, and mounted for microscopy.

Immunoreactivity (both quantitative and qualitative) in the tissue biopsies are identified in small and medium-sized vessels under low power (40× or 100×) to identify areas with microvessel density (MVD). Only sections that show presence of tumor (as determined by H&E stained slides) are evaluated for MVD. Microvascular density per area (mean MVD/mm²±SD) are then determined under higher magnification, at 200× or 400× as previously described (Wood, L. S. et al. Horiz. In Cancer Therap., 3:24-25 (2002); Overmoyer, B. et al. Proc. ASCO, 20:99a (2001); Hlatky, L. et al. J. Natl. Cancer Inst., 94:883-893 (2002); Brem, S. et al. J. Natl. Cancer Inst., 48:347-56 (1972); Weidner, N. et al. N. Engl. J. Med., 324:1-8 (1991)). A single microvessel is considered as an endothelial cell or cell cluster with brown reaction product that is distinct from adjacent microvessels, tumor cells, or other tissue elements. Mature versus immature vessels is determined by evaluating vessels stained with or without the smooth muscle actin stain (Wood, L. S. et al. Horiz. In Cancer Therap., 3:24-25 (2002); Darland, D. C. et al. J. Clin. Invest., 103:157-8 (1999)). Tissue cellularity and collagen deposition is used to qualitatively assess the tissue reaction. Mitotic counts are determined by the brown reaction, nuclear Ki67 positive cells. Appropriate statistical analyses include Kruskal-Wallis or Mann-Whitney nonparametric tests.

### Example 9

### Vasoreactivity of Normal Prostatic Vessels Produced by Silfenadil and Pseudoephedrine: Potential for Improved Diagnostic Imaging

Blood flow of the normal prostate has received limited attention in the literature, except for establishment of a baseline comparison for the evaluation of inflammatory and cancerous processes. While absolute quantitative methods for precise measurement of prostatic blood flow have yet to be developed, the most widely accepted method is the semi-quantitative method DCE MRI (dynamic contrast enhanced magnetic resonance imaging).

DCE MRI of the prostate, as reported in the literature, is performed by acquiring repeat MRI images over the prostate during the intravenous injection of gadolinium contrast material. The image data acquired over a local area of interest can be used to plot a curve of the signal intensity over time. Such curves semiquantitatively reflect the blood flow and vessel density in the area of interest (quantitative measurement is not possible because of the variable paramagnetic effects related to concentration variation).

Numerous authors have used this method to show differential enhancement between the normal gland and tumor. The angiogenesis of prostatic cancer stimulates increased vessel growth and density (maximum vessel density) as compared to normal tissue. Accordingly, DCE MRI of the tumor tissue demonstrates increased signal intensity reflecting the increased number of tumor blood vessels. The shortcoming of the current technique is the overlap of findings with prostatitis and cancer, because both can show increased signal intensity due to increased blood flow. A potential method of improving the differentiation of the two may the use of vasoactive drugs which can induce characteristic changes in normal and tumor vessels.

This case demonstrates the vasoreactivity of normal prostate vessels produced by the vasodilator, 5-phosphodiesterase inhibitor, silfenadil, and the alpha vasoconstrictor, pseudoephedrine. This vasomodulated change in blood measured on DCE MRI and can form the basis for improved cancer imaging of the prostate. This is the first description of the effects of these drugs on prostate blood flow.

### Case History

A 59 year old white male with no history of prostate cancer and normal PSA, volunteered for multiple DCE MRI exams of the prostate to evaluate vasoactive modulation of the normal blood flow. Three separate examinations were performed several weeks apart. The studies were performed on a Siemens 1.5T Symphony scanner. The DCE MRI examination included, Siemens's tfiperf, (inversion fisp) sequence. TR=3000, TE=1.27, TI=400 flip angle=50. Gadolinium Veresetamide 33.9 mg, injection at 2 cc/sec for a total of 20 cc. Images were obtained every 3 seconds for a total period of 5 minutes and every minute thereafter for 15 minutes.

All three studies were performed with identical MRI sequences and parameters. The first study was performed without a vasoactive drug but the second and third study were repeated with administration of a vasoactive drug, silfenadil or pseudoephedrine. The second DCE MRI study was performed 1 week later, the gadolinium injection was performed after the oral ingestion of 25 mg of silfenadil one hour prior to the study. The third DCE MRI was performed identical to the second study with the oral administration of 25 mg of silfenadil one hour before the study, and the administration of 60 mg of pseudoephedrine 20 minutes preceding the gadolinium injection. The absorption rate of each drug is quite predictable according to the literature so the initial part of the study performed before the pseudoephedrine was vasodilated and the subsequent study had vasodilator combined with a vasoconstrictor.

Data analysis was performed using ANALYZE data management software (Analyze Direct, Inc., Lenexa, Kans., 66215, USA) and Excel (Microsoft, Seattle, Wash.).

Data graphs were normalized to the blood flow of the iliac artery. The data was acquired during the five minute period following intravenous gadolinium injection and thereby provides a semiquantitative assessment of blood flow characteristics, see below data discussion.

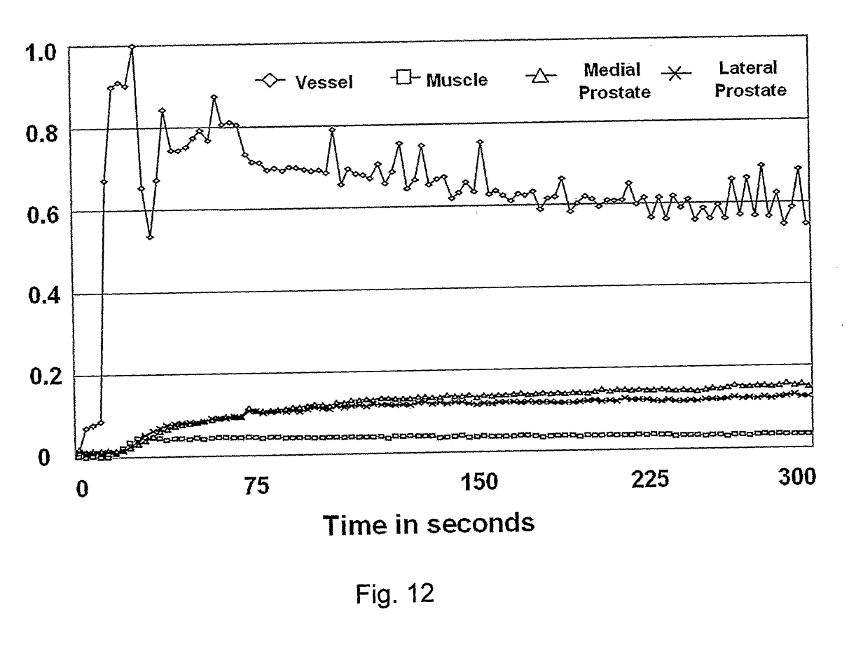

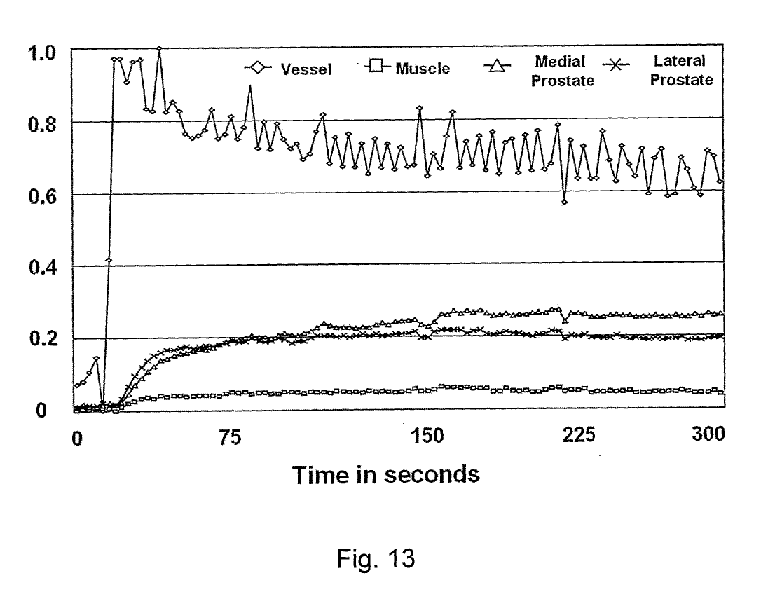

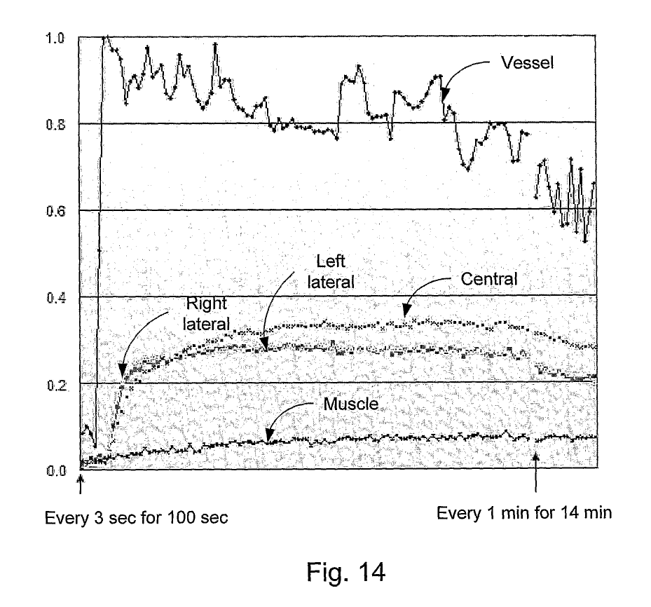

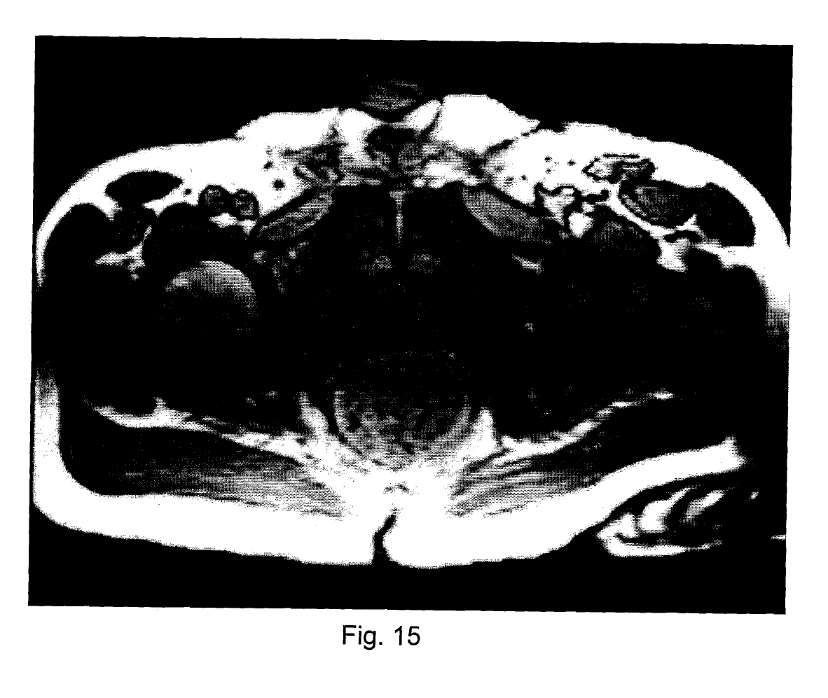

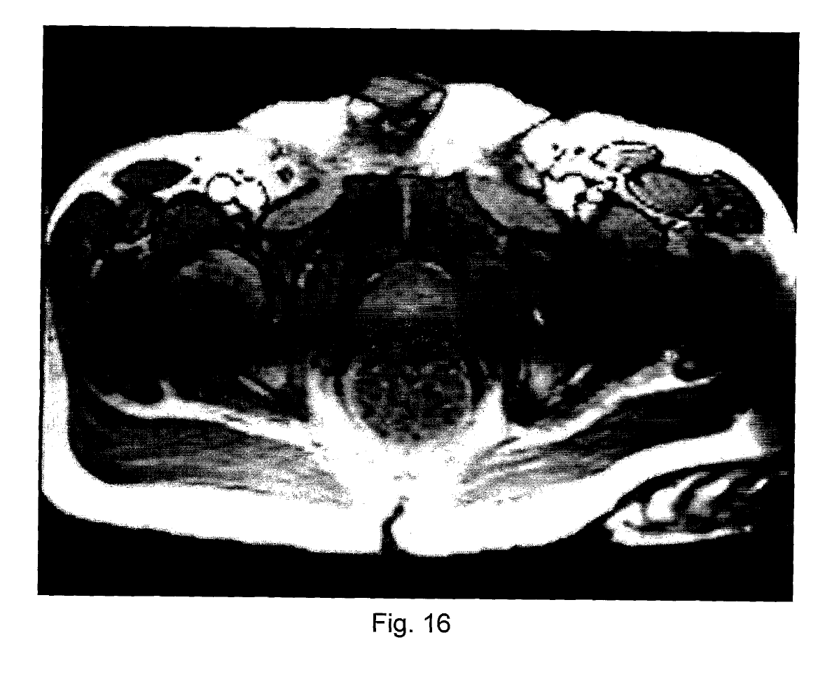

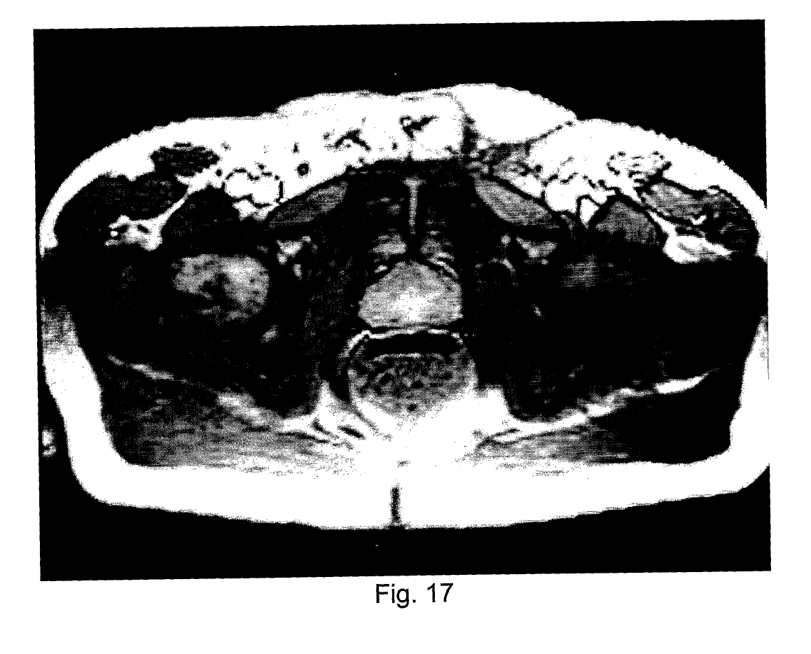

The results of the intensity curves are seen in FIGS. 12, 13, and 14. Clinical MRI images showing enhancement after silfenadil are noted in FIGS. 15, 16, and 17. Comparing the baseline intensity flow curve with the sildenafil curve, FIGS. 12 and 13, several observations can be made. The enhancements of the lateral and central portions of the prostate on the two studies begins at 21 seconds and show significant differences in the amount of total enhancement and relative enhancement between the two lobes. Most importantly, comparing the baseline enhancement curve (without sildenafil) to the vasodilated enhancement curve with silfenafil, there is more than 70% increase throughout the entire scan period of 5 minutes (300 seconds). Also of great interest is the differential enhancement seen on the comparison studies of the medial and lateral lobes during the early phase and later phase of contrast. Between 21 seconds and 72 seconds the medial lobe increased 73% and the lateral lobe increased by 78% on the silfenadil study compared to the baseline study. On the baseline study without sildenafil there was essentially no difference in the enhancement between the medial and lateral lobes during the early phase. Comparing the baseline scan and the sildenafil enhanced scan after 72 seconds shows increased enhancement of the medial lobe compared to the lateral lobes but to a greater degree than on the baseline.

The DCE MRI study with silfenadil and pseudoephedrine is equally interesting. Firstly, the enhancement pattern of the medial and lateral lobes of the prostate are again demonstrated during the early phase of the study. Most interesting is the changes in blood flow which occur in the later phases. After 14 minutes there was a reduction of about 20% in the enhancement. At this phase of the gadolinium equilibrium, the intensity depends upon the intravascular and extravascular concentration. Because the diffusion to the extravascular space would not be affected, the reduction in intensity must be secondary to vascular constriction in the gland.

### Discussion

Our discovery of the vasoactive effects of sildenafil and pseudoephedrine on normal prostate vessels and the ability to semiquantify it during DCE MRI scanning has two significant implications. Firstly, it demonstrates that the same vascular receptor sites for sildenafil (5-phosphodiesterase inhibitor) and the alpha sites for pseudoephedrine are present in the prostate vessels and penis. Secondly. the vasodilatation and increased enhancement of the normal prostate vessels detectable by DCE MRI has the potential for improved diagnosis of prostatic cancer and inflammation.

While others have confirmed the effect of 5-phosphodiesterase inhibitors, such as silfenadil to produce vasodilation and pseudoephedrine to produce vasoconstriction of the penile arterioles, the effect on the prostate has not been previously documented. We intuitively anticipated this observation because the prostate gland produces approximately 30% of the semen volume ejaculated during sexual climax. In cases of priaprism induced by these inhibitors the oral administration of pseudoephedrine has been used to reduce blood flow by constriction of penile vessels to alleviate the erection.

This confirmation of the vasoreactivity of normal prostate vessels provides the basis for exploiting the reported structural and functional differences of normal and prostate tumor vessels. While normal vessels have smooth muscle pericytes which are reactive, tumor vessels lack such cells. Using quantitative immunohistochemical staining (CD34) endothelial cells and alpha SMA for mural cells) of human prostate tumor it has been noted that prostatic tumor vessel pericytes were lacking in 70% of vessels. Additional rationale to expect non-reactivity from tumor vessels has also been noted.

The paradoxical indirect effect of normal vessel vasoactive response on tumor blood flow was demonstrated has been demonstrated. Their animal model data demonstrated that when normal vessels react by dilating or constricting, the opposite effect is paradoxically induced in tumor vessels. When normal vessels dilate, the blood is “pulled” away from tumor vessels producing a “steal” phenomenon. When normal vessels constrict, the blood is “pushed” to the tumor vessels increasing their relative blood flow. We have observed that in several clinical human cases, they observed a similar paradoxical “steal” of blood flow from tumor to normal, in liver and kidney cancer, on DCE MRI imaging. This effect was demonstrated by comparing signal intensity/time curves on baseline and vasodilator modulated studies.

With the demonstrated vasoreactivity of normal prostate vessels, we propose developing a DCE MRI examination which exploits the vasomodulation differences between normal and cancer vessels. To this end, silfenadil and pseudoephedrine either as single or combined (temporal differences of administration) would be well suited because of their overall high safety margin. Silfenadil's low incidence of side effects as a treatment for erectile disfunction is well known and it appears to not adversely affect tumor growth. It has been reported that silfenadil did not promote tumor cell growth in an orthotopic prostate cancer model, so any concern about promoting tumor growth is minimal.

To our knowledge this is the first report of increased enhancement and blood flow of the normal prostate, produced by a 5-phosphodiesterase inhibitor. silfenadil and decreased enhancement induced by pseudoephedrine. Because of the vaso reactivity of the normal prostate vessels to such agents, we believe vasoactive drugs may form the basis for a new imaging approach to differentiate cancer from normal and inflammatory tissues.

From the above description of the invention, those skilled in the art will perceive improvements, changes and modifications. Such improvements, changes and modifications within the skill of the art are intended to be covered by the appended claims.

Having described the invention, we claim:

1. A method for detecting abnormal biological tissue, the method comprising:
administering an amount of a vasoactive agent effective to modify the blood flow rate in a tissue of interest; and
determining, following administration of the vasoactive agent, whether the blood flow rate in the tissue has increased or decreased in comparison to blood flow rate in normal tissue.

2. The method of claim 1, wherein administering the vasoactive agent includes injecting the vasoactive agent into the vasculature of a patient.

3. The method of claim 1, wherein the vasoactive agent is a vasodilatory agent.

4. The method of claim 1, wherein the vasoactive agent is a vasoconstrictive agent.

5. The method of claim 1, the blood flow rate in the tissue of interest being determined by generating at least one first image of the tissue of interest prior to administration of the vasoactive agent, generating at least one second image of the tissue of interest after administration of the vasoactive agent, and comparing first images and the second images.

6. The method of claim 5, first image and the second image being compared to identify any local variations in the change in signal intensity resulting from vascular volume changes induced by the vasoactive agent.

7. The method of claim 5, the first images and the second images being generated by at least one of computed tomography (CT), magnetic resonance (MR), ultrasound, and X-ray.

8. The method of claim 3, a decrease in the blood flow rate of the tissue of interest indicates abnormal biological tissue.

9. The method of claim 4, an increase in the blood flow rate of the tissue of interest indicates abnormal biological tissue.

10. The method of claim 1, the abnormal biological tissue includes neoplastic tissue.

11. A method of detecting abnormal biological tissue in a patient, the method comprising:
determining the blood flow rate in a tissue of interest prior to administration of a vasoactive agent;
administering an amount of the vasoactive agent effective to modify the blood flow rate in the tissue of interest; and
determining the blood flow rate in the tissue of interest following administration of the vasoactive agent.

12. The method of claim 11, the blood flow rate in the tissue of interest being determined prior to and following administration of the vasoactive agent by imaging the tissue of interest.

13. The method of claim 10, the vasoactive agent including at least one of a vasoconstrictive agent and vasodilatory agent selected from the group consisting of carbonic anhydrase inhibitors, caffeine citrate, organic nitrates, glyceryl trinitrate, pentaerythritol tetranitrate, hydralazine, sildenafil citrate, minoxidil, diazoxide, sodium nitroprusside, isosorbide dinitrate, isosorbide mononitrate, cilostazol, papaverine, dipyridamole, oxyfedrine hydrochloride (HCl), diltiazem HCl, tolazoline HCl, hexobendine, bamethan sulfate, sulfonamide derivatives, phenylephrine HCl, pitressin, pseudoephedrine, angiotensin, vasopressin, levonordefrin, epinephrine, naphazoline nitrate, tetrahydrozoline HCl, oxymetazoline HCl, tramazoline HCl, lypressin, and combinations thereof.

14. The method of claim 12, imaging of the tissue of interest being performed by at least one of computed tomography, magnetic resonance, ultrasound, and X-ray.

15. The method of claim 141, wherein the abnormal biological tissue includes neoplastic tissue.

16. The method of claim 15, wherein the abnormal biological tissue comprises at least one of abnormal tissue in the liver and renal tissue.

17. A method for distinguishing neoplastic and normal tissue in a patient, the method comprising:
administering an amount of a vasoactive agent effective to modify the blood flow rate in a tissue of interest; and
determining, following administration of the vasoactive agent, whether the blood flow rate in the tissue has increased or decreased in comparison to blood flow rate in normal tissue.

18. The method of claim 17, the vasoactive agent comprising acetazolamide.

19. The method of claim 17, the blood flow rate in the tissue of interest being determined by generating at least one first image of the tissue of interest prior to administration of the vasoactive agent, generating at least one second image of the tissue of interest after administration of the vasoactive agent, and comparing first images and the second images.

20. The method of claim 19, the images of the tissue of interest being generated by at least one of computed tomography, magnetic resonance, ultrasound, and X-ray.
